(19) United States
(12) Patent Application Publication
(10) Pub. No.: US 2021/0378992 A1
(43) Pub. Date: Dec. 9, 2021

(54) METHODS FOR TREATING A SUBTYPE OF SMALL CELL LUNG CANCER

(71) Applicant: Auransa Inc., Palo Alto, CA (US)

(72) Inventors: Johan GRAHNEN, Palo Alto, CA (US); Pek Yee LUM, Palo Alto, CA (US); Zhewei SHEN, Palo Alto, CA (US); Victor SOLOVYEV, Palo Alto, CA (US); Hak Jin CHANG, Harvard, MA (US)

(21) Appl. No.: 17/286,396

(22) PCT Filed: Oct. 18, 2019
(86) PCT No.: PCT/US2019/057080

§ 371 Date: Apr. 16, 2021

Related U.S. Application Data
(60) Provisional application No. 62/748,229, filed on Oct. 19, 2018.

(51) Int. Cl. A61K 31/155; G01N 33/574; C12Q 1/6886; A61K 31/357; A61K 33/243; A61P 35/00

(52) U.S. Cl. CPC A61K 31/155; G01N 33/57423; C12Q 1/6886; C12Q 2600/106; A61K 33/243; A61P 35/00; C12Q 2600/158; A61K 31/357

(57) ABSTRACT

This disclosure provides methods for treating a subject having small cell lung cancer by determining expression levels of biomarkers highly correlated with a subtype of small cell lung cancer that are sensitive to treatment with pentamidine or a pharmaceutically acceptable salt thereof. The methods are drawn to determining a predictive gene expression profile of a subtype of small cell lung cancer and treating the subject with an effective amount of pentamidine or a pharmaceutically acceptable salt of pentamidine as a chemotherapy agent. The methods generally involve treatment of a subtype of small cell lung cancer predicted to be a responder to pentamidine or a pharmaceutically acceptable salt of pentamidine.

### CROSS REFERENCE TO RELATED APPLICATION

This application claims priority to U.S. Provisional Application Ser. No. 62/748,229, filed Oct. 19, 2018, which is hereby incorporated by reference in its entirety.

### BACKGROUND

Pentamidine, 1,5-Bis(4-amidinophenoxy)pentane, came into medical use in 1937 and is currently on the World Health Organization's List of Essential Medicines as an antiprotazoal/antifungal agent used for the treatment of various infectious diseases such as African trypanosomiasis, leishmaniasis, babesionsis, and Pneumocystis carinii pneumonia. Pentamidine has been also proposed to have anticancer properties through its inhibitory effects on PRLs (phosphatase of regenerating liver family), the endo-exonuclease activity, and the interaction between S100B and p53. This compound has been shown to preferentially bind to DNA in the minor groove of AT-rich domains.

About 14% of all new cancer cases worldwide are lung cancers. In 2017, an estimated 222,000 adults (117,000 men and 105,000 women) in the United States are diagnosed with lung cancer. This number includes people diagnosed with small cell lung cancer (SCLC) and non-small cell lung cancer (NSCLC). Each year, more people die from lung cancer than from colon, breast and prostate cancers combined. Particularly, the five-year survival rate for SCLC patients diagnosed with Stage I SCLC is about 31%. For Stages II and III SCLC, the survival rate significantly drops to about 19% and 8%, respectively. For hard-to-treat metastatic SCLC, the 5-year survival rate is only about 2% or less. Currently available treatments against SCLC include chemotherapy using an etoposide-based drug (e.g., Toposar or Vepesid) and cisplatin in combination with radiation therapy, depending on the stage of SCLC. The high mortality rate, however, continues to rise largely due to the increased number of non-responders to the existing therapy.

Given the grave consequences and mortality rates in SCLC, there is a dire need for an effective drug to treat small cell lung cancer. Of particular importance is the identification and development of a drug precisely matched with a subtype of SCLC predicted to be responsive to the drug as such an approach can significantly improve the clinical outcome and survival rate of SCLC.

### BRIEF DESCRIPTION OF THE DRAWINGS

FIG. 1 depicts tumor volume of LU5243 for vehicle (saline), 10 mg per kg of pentamidine, and 20 mg per kg of pentamidine in mice. Mean absolute tumor volumes ±SEM; individual tumor volumes; and study day 1 corresponding to day of first dose.

FIG. 2 depicts body weight of LU5243 for vehicle (saline), 10 mg per kg of pentamidine, and 20 mg per kg of pentamidine in mice. Mean absolute body weight ±SEM; individual body weights; and study day 1 corresponding to day of first dose.

FIG. 3 depicts tumor volume of LU5141 for vehicle (saline), 10 mg per kg of pentamidine, and 20 mg per kg of pentamidine in mice. Mean absolute tumor volumes ±SEM; individual tumor volumes; and study day 1 corresponding to day of first dose.

FIG. 4 depicts body weight of LU5141 for vehicle (saline), 10 mg per kg of pentamidine, and 20 mg per kg of pentamidine in mice. Mean absolute body weight ±SEM; individual body weights; and study day 1 corresponding to day of first dose.

### BRIEF SUMMARY

This disclosure involves methods useful for treating patients suffering from small cell lung cancer with pentamidine or a pharmaceutically acceptable salt thereof (e.g., pentamidine isethionate, pentamidine gluconate, or pentamidine mesylate). By identifying a subtype of small cell lung cancer sensitive to treatment with pentamidine. The invention is based, at least in part, on the discovery that an increased and/or decreased level of expression of certain biomarkers identified herein are indicative that the subject will respond to treatment with pentamidine or a pharmaceutically acceptable salt thereof (e.g., pentamidine isethionate, pentamidine gluconate, or pentamidine mesylate) and the application of the discovery to develop a method of treating small cell lung cancer. For example, a high expression of one or more biomarkers listed in Table 1 in a small cell lung tumor sample is predictive that the subject will respond to treatment with pentamidine or a pharmaceutically acceptable salt thereof (e.g., pentamidine isethionate, pentamidine gluconate, or pentamidine mesylate) in a manner such that the treatment reverses diseased expression profiles of these genes. Further, a low level of expression of a certain biomarker, for example, one or more biomarkers listed in Table 2 in a SCLC tumor sample is indicative of that the subject will respond to treatment with pentamidine or a pharmaceutically acceptable salt thereof (e.g., pentamidine isethionate, pentamidine gluconate, or pentamidine mesylate). The biomarker expression profile is predicted to reverse in a small cell lung cancer tumor of the subject, upon the treatment with pentamindine or a pharmaceutically acceptable salt thereof (e.g., pentamidine isethionate, pentamidine gluconate, or pentamidine mesylate).

Accordingly, in one aspect, the present invention provides a method of treating a subject having small cell lung cancer by: (i) assaying a small cell lung cancer sample derived from said subject to determine the level of expression in said sample of at least one biomarker selected from the group of biomarkers listed in Table 1 and optionally at least one biomarker from Table 2; (ii) detecting a high level of expression of said at least one biomarker in Table 1 and optionally a low level of expression of said at least one biomarker in Table 2 in said sample relative to a normal control; and (iii) administering a therapeutically effective amount of pentamidine or a pharmaceutically acceptable salt thereof, to said subject. Preferably, said at least one biomarker selected from Table 1 is INSM1. In various embodiments, the level of expression of at least 2, at least 3 or at least 4 biomarkers selected from the group of biomarkers listed in Table 1 and 2 is determined. For examples, the predictive gene expression signature may include INSM1 and at least one additional biomarkers selected from the group consisting of GADD45G, STAT6, SMAD3, C1QL1 and RNF43. In some embodiments, the predictive gene expression signature may include at least three biomarkers comprising INSM1 and two or more additional biomarkers selected from the group consisting of GADD45G, STAT6, SMAD3, C1QL1, and RNF43.

In one aspect, the present invention also provides a method for determining whether pentamidine or a pharmaceutically acceptable salt thereof, can be used to treat a subject having small cell lung cancer by assaying a small cell lung cancer sample derived from the subject to determine the level of expression in said sample of at least one biomarker selected from the group of biomarkers listed in Table 1, Table 2 and/or Table 3. In one embodiment, a high level of expression of at least one biomarker in Table 1, optionally in combination with a low level of expression of one or more biomarkers listed in Table 2 are highly predictive that the subject suffers from a subtype of small cell lung cancer which will respond to treatment with pentamidine or a pharmaceutically acceptable salt thereof, and that treatment with pentamidine or a pharmaceutically acceptable salt thereof will be efficacious in treating the subject suffering from small cell lung cancer. In another embodiment, a high level of expression of at least one biomarker in Table 1, in combination with a low level of expression of one or more biomarkers listed in Table 2 and optionally a high level of expression of at least one biomarker in Table 3 are highly predictive that the subject suffers from a subtype of small cell lung cancer which will respond to treatment with pentamidine or a pharmaceutically acceptable salt thereof. In some embodiments, the treatment with pentamidine or a pharmaceutically acceptable salt thereof effectively reverses the biomarker gene expression signature of the selected biomarkers in SCLC tumors to an expression level similar to one observed in corresponding non-tumor tissues (e.g., normal controls). Preferably, the at least one biomarker selected from Table 1 is INSM1.

In one aspect, the levels of expression of biomarkers are determined by detecting mRNA levels. In another aspect, the levels of expression of biomarkers are determined by detecting protein levels produced from the biomarker genes.

In one embodiment, a pharmaceutically acceptable salt of pentamidine is pentamidine isethionate. In another embodiment, a pharmaceutically acceptable salt of pentamidine is pentamidine gluconate. In another embodiment, a pharmaceutically acceptable salt of pentamidine is pentamidine mesylate.

In some aspects, pentamidine or a pharmaceutically acceptable salt of pentamidine can be administered in combination with additional chemotherapy agent, e.g., etoposide, cisplatin, oxaliplatin, gemcitabine, irinotecan, and taxol.

In some aspects, pentamidine or a pharmaceutically acceptable salt of pentamidine can be administered intravenously, intramuscularly, subcutaneously, orally, or by inhalation.

In some aspects, pentamidine or a pharmaceutically acceptable salt thereof is administered to a subject at a daily dose of about 0.5 mg per kg to about 30 mg per kg.

Provided herein are methods of treating a subject suffering from small cell lung cancer expressing a high level of at least one biomarker selected from the group consisting of INSM1, GADD45G, and C1QL1, the methods comprising administering a therapeutically effective amount of pentamidine or a pharmaceutically acceptable salt thereof, to the subject.

Also provided herein are methods of treating a subject suffering from small cell lung cancer expressing a high level of at least one biomarker selected from the group consisting of INSM1, GADD45G, and C1QL1 and a low level of at least one biomarker selected from the group consisting of STAT6, SMAD3, and RNF43, the methods comprising administering a therapeutically effective amount of pentamidine or a pharmaceutically acceptable salt thereof, to the subject.

Also provided herein are methods of treating a subject suffering from small cell lung cancer, the methods comprising administering a therapeutically effective amount of pentamidine or a pharmaceutically acceptable salt thereof, to the subject, wherein expression of a high level of at least one biomarker selected from the group consisting of INSM1, GADD45G, and C1QL1 by the small cell lung cancer is used as a basis for selecting the subject to receive treatment.

In some embodiments, expression of a low level of at least one biomarker selected from the group consisting of STAT6, SMAD3, and RNF43 by the small cell lung cancer is used as a basis for selecting the subject to receive treatment. In some embodiments, the method further comprises determining the level of expression of the at least one biomarker selected from the group consisting of INSM1, GADD45G, and C1QL1. In some embodiments, the method further comprises determining the level of expression of at least one biomarker selected from the group consisting of STAT6, SMAD3, and RNF43 by the small cell lung cancer.

Also provided herein are methods of treating a subject suffering from small cell lung cancer, the methods comprising a) determining the level of expression by the small cell lung cancer of at least one biomarker selected from the group consisting of INSM1, GADD45G, and C1QL1; and b) administering a therapeutically effective amount of pentamidine or a pharmaceutically acceptable salt thereof, to said subject, wherein the small cell lung cancer expresses a high level of the at least one biomarker selected from the group consisting of INSM1, GADD45G, and C1QL1.

Also provided herein are methods of identifying a subject suitable for small cell lung cancer treatment, the methods comprising determining the level of expression of at least one biomarker selected from the group consisting of INSM1, GADD45G, and C1QL1 by the small cell lung cancer, wherein the subject is identified as suitable for small cell lung cancer treatment with a therapeutically effective amount of pentamidine or a pharmaceutically acceptable salt thereof if the small cell lung cancer expresses a high level of the at least one biomarker selected from the group consisting of INSM1, GADD45G, and C1QL1.

In some embodiments, the method comprises determining the level of expression of INSM1. In some embodiments, the method comprises determining the level of expression of GADD45G. In some embodiments, the method comprises determining the level of expression of C1QL1. In some embodiments, the small cell lung cancer expresses a high level of INSM1. In some embodiments, the small cell lung cancer expresses a high level of GADD45G. In some embodiments, the small cell lung cancer expresses a high level of C1QL1. In some embodiments, the high level is a level greater than a control level.

In some embodiments, the method further comprises determining the level of expression of at least one biomarker selected from the group consisting of STAT6, SMAD3, and RNF43 by the small cell lung cancer. In some embodiments, the method comprises determining the level of expression of STAT6. In some embodiments, the method comprises determining the level of expression of SMAD3. In some embodiments, the method comprises determining the level of expression of RNF43. In some embodiments, the small cell lung cancer expresses a low level of at least one biomarker selected from the group consisting of STAT6, SMAD3, and RNF43. In some embodiments, the small cell lung cancer expresses a low level of STAT6. In some embodiments, the small cell lung cancer expresses a low level of SMAD3. In some embodiments, the small cell lung cancer expresses a low level of RNF43. In some embodiments, the low level is a level that is lower than a control level or undetectable.

In some embodiments, the small cell lung cancer expresses a high level of expression at least one biomarker from the group consisting of APLP1, SEZ6L2, LHX2, SMAD9, DPF1, NBEA, MPPED1, PCSK1N, WSCD1, RALYL, H2AFX, TUBB3, GABRD, HOXB8, DLX2, CLDN11, CKB, ID4, SBNO2, FZD9, AKR1C3, MTAP, SNCAIP, DLX5, CADPS, DGKB, LFNG, CER1, KIAA0319, VWA5B2, SLC22A17, FOXA2, HNRNPA0, HELLS, NRTN, and SOX21. In some embodiments, the method further comprises detecting a high level of expression at least one biomarker from the group consisting of APLP1, SEZ6L2, LHX2, SMAD9, DPF1, NBEA, MPPED1, PCSK1N, WSCD1, RALYL, H2AFX, TUBB3, GABRD, HOXB8, DLX2, CLDN11, CKB, ID4, SBNO2, FZD9, AKR1C3, MTAP, SNCAIP, DLX5, CADPS, DGKB, LFNG, CER1, KIAA0319, VWA5B2, SLC22A17, FOXA2, HNRNPA0, HELLS, NRTN, and SOX21.

In some embodiments, determining the level of expression comprises assaying a nucleic acid or protein level in a small cell lung cancer sample from said subject. In some embodiments, the small cell lung cancer sample is a fluid sample comprising circulating tumor cells or cell-free nucleic acids. In some embodiments, the small cell lung cancer sample is a tissue or a cell sample. In some embodiments, the level of expression of said biomarker is determined by detecting mRNA levels. In some embodiments, the level of expression of said biomarkers is determined at the protein level. In some embodiments, the presence of the protein is detected using an antibody that binds to the protein. In some embodiments, the antibody is labeled. In some embodiments, the level of expression of said biomarker is determined by immunoassay, a western blot assay, immunofluorimetry, ELISA assay, electrochemiluminescence assay, or immunoprecipitation.

In some embodiments, the pharmaceutically acceptable salt of pentamidine is pentamidine isethionate. In some embodiments, the pharmaceutically acceptable salt of pentamidine is pentamidine mesylate. In some embodiments, the pharmaceutically acceptable salt of pentamidine is pentamidine gluconate.

In some embodiments, the small cell lung cancer is Stage I, II or III small cell lung cancer. In some embodiments, the subject is a human patient. In some embodiments, the pentamidine or pharmaceutically acceptable salt thereof is administered to the subject orally. In some embodiments, the pentamidine or pharmaceutically acceptable salt thereof is administered to the subject by inhalation. In some embodiments, the pentamidine or pharmaceutically acceptable salt thereof is administered to the subject intravenously.

In some embodiments, the pentamidine or a pharmaceutically acceptable salt thereof is administered to the subject with at least one or more additional anticancer agents. In some embodiments, the additional anticancer agent is etoposide. In some embodiments, the additional anticancer agent is cisplatin. In some embodiments, the pentamidine or the pharmaceutically acceptable salt thereof is administered to said subject at a daily dose of about 0.5 mg per kg to about 30 mg per kg.

The present invention also contemplates kits comprising pentamidine or a pharmaceutically acceptable salt thereof and biomarker test materials described below. Also provided herein are kits comprising: a) a pentamidine or a pharmaceutically acceptable salt thereof; b) testing materials for predictive gene expression signature of one or more biomarkers for a subtype of small cell lung cancer sensitive to treatment with pentamidine or pharmaceutically acceptable salt thereof; and c) an instruction for use.

### DETAILED DESCRIPTION

Unless otherwise defined herein, scientific and technical terms used in connection with the present disclosure shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms, for example, those characterized by “a” or “an”, shall include pluralities, e.g., one or more biomarkers. In this application, the use of “or” means “and/or”, unless stated otherwise. Furthermore, the use of the term “including,” as well as other forms of the term, such as “includes” and “included,” is not limiting. Also, terms such as “element” or “component” encompass both elements and components comprising one unit and elements and components that comprise more than one unit unless specifically stated otherwise.

As used herein, the term “biomarker” is intended to include a substance that is used as an indicator of a biological state and includes for example, genes (or portions thereof), mRNAs (or portion thereof), miRNAs (microRNAs or portions thereof), and proteins (or portions thereof). A “biomarker expression profile” or “biomarker expression signature” is intended to refer to a quantitative or qualitative summary of the expression of one or more biomarkers in a tumor of a subject, such as in comparison to a standard or a control.

As used herein, the phrase “predictive gene expression signature” or “predictive gene expression profile” refers to expression levels of one or more biomarkers of the present invention in a subject that are indicative of responsiveness to treatment with pentamidine or a pharmaceutically acceptable salt thereof. For example, a high level of expression of at least 1, at least 2, at least 3, at least 4, at least 5, at least 6, at least 7, at least 8, at least 9 or at least 10 biomarkers from Table 1, Table 2, or Table 3 in a subject may constitute a gene expression signature or gene expression profile that indicates that the subject will respond positively to treatment with pentamidine or a pharmaceutically acceptable salt thereof (e.g., pentamidine isethionate, pentamidine gluconate, or pentamidine mesylate). Any combination of two or more markers from Table 1, Table 2, or as shown in, for example, Table 3, may constitute a predictive gene expression signature or predictive gene expression profile of the invention. In another example, the expression of at least 1, at least 2, at least 3, at least 4, at least 5, at least 6, at least 7, at least 8, at least 9 or at least 10 biomarkers from Table 1, Table 2 or Table 3 above particular threshold as in a high level of expression or under particular threshold levels as in a low level of expression constitutes a gene expression signature or gene expression profile that indicates that the subject will respond to positively to treatment with pentamidine or a pharmaceutically acceptable salt thereof (e.g., pentamidine isethionate, pentamidine gluconate, or pentamidine mesylate).

A “high level of expression” of the biomarker, for example, a biomarker selected from the group of biomarkers listed in Table 1 or Table 3, refers to a level of expression of the biomarker in a sample (e.g., a sample derived from a subject) that correlates with sensitivity to pentamidine or a pharmaceutically acceptable salt thereof (e.g., pentamidine isethionate, pentamidine gluconate, or pentamidine mesylate). This can be determined by comparing the level of expression of the selected biomarker in the test sample with that of a suitable control (e.g., normal lung cells). The level of elevated expression can be at least 2 times to at least 50 times greater than expression levels measured in one or more controls. The level of increased expression can be at least 3 times to at least 40 times greater than expression levels measured in one or more controls. The level of elevated expression can be at least 4 times greater than an expression level measured in a control. The level of elevated expression can be at least 5 times greater than an expression levels measured in a control. The level of elevated expression can be at least 10 times greater than an expression level measured in a control. The level of elevated expression can be at least 15, 20, 30, 40 or 50 times greater than an expression level measured in a control. Therefore, a high level of expression will be above or within the higher levels of the range descried above.

A “low level of expression” of the biomarker, for example, a biomarker selected from the group of biomarkers listed in Table 2 refers to a level of expression of the biomarker in a test sample (e.g., a sample derived from a subject) that correlates with sensitivity to pentamidine or a pharmaceutically acceptable salt thereof (e.g., pentamidine isethionate, pentamidine gluconate, or pentamidine mesylate). This can be determined by comparing the level of expression of one or more biomarkers in the test sample with that of a suitable control (e.g., normal lung cells). A “low level of expression” may include lack of detectable expression of the biomarkers. A “low level of expression” can also include levels similar to or lower than those measured in controls (e.g., normal lung cells). A “low level of expression” can be at least 95%, 90%, 80%, 70%, 60%, 50%, 40%, 30%, 20%, 10%, 5%, 1% lower than expression levels measured in a control (e.g., normal lung cells). As such, in some embodiments, the level of expression of the biomarker is determined relative to a control sample, for example, the level of expression of the biomarker in a corresponding normal lung tissue or cell (e.g., a range determined from the levels of expression of the biomarker observed in normal lung tissue samples). Therefore, “low level of expression” will fall below or within the low levels of the range observed in normal lung tissue samples.

In some embodiments, the level of expression of the biomarker is determined relative to a control sample, such as the level of expression of the biomarker in samples, for examples, tumor samples, circulating tumor cells (CTCs), etc. from other subjects. For example, the level of expression of the biomarker in samples from other subjects can be determined to define levels of expression which correlate with sensitivity to treatment with pentamidine or a pharmaceutically acceptable salt thereof (e.g., pentamidine isethionate, pentamidine gluconate, or pentamidine mesylate), and the level of expression of the biomarker in the sample from the subject of interest is compared to these levels of expression, wherein a high, comparable, or lower level of expression in the sample from the subject is indicative of a “high level or expression” or “low level of expression” of the biomarker in the sample. In another example, the level of expression of the biomarker in samples (e.g., tumor samples, circulating tumor cells, etc.) from other subjects can be determined to define levels of expression which correlate with resistance or non-responsiveness to treatment with pentamidine, or a pharmaceutically acceptable salt thereof (e.g., pentamidine isethionate, pentamidine gluconate, or pentamidine mesylate), and the level of expression of the biomarker in the sample from the subject of interest is compared to these levels of expression, wherein a high level of expression or a lower level of expression in the sample from the subject is indicative of a “high level of expression” or “low level of expression” of the biomarker in the sample.

The term “control level” or “known standard level” can refer to an accepted or predetermined expression level of the biomarker, for example, a biomarker selected from the group of biomarkers listed in Table 1 which is used to compare expression level of the biomarker in a sample derived from a subject. In one embodiment, the control expression level of the biomarker is the average expression level of the biomarker in samples derived from a population of subjects. For example, the control expression level can be the average expression level of the biomarker in small cell lung cancer cells derived from a population of subjects with small cell lung cancer. The population may be subjects who have not responded to treatment with pentamidine, or a pharmaceutically acceptable salt thereof (e.g., pentamidine isethionate, pentamidine gluconate, or pentamidine mesylate). In some embodiments, the control level may constitute a range of expression of the biomarker in one or more healthy subjects. In some embodiments, the control level may constitute a range of expression of the biomarker in a normal lung tissue. For example, the control level may constitute a range of expression of the biomarker in tumor samples from a variety of subjects having small cell lung cancer, as described above. As further information becomes available as a result of routine performance of the methods described herein, population-average values for “control” level of expression of the biomarkers of the present invention may be used. In other embodiments, the “control” level of expression of the biomarkers may be determined by determining expression level of the respective biomarker in a subject sample obtained from a subject before the suspected onset of small cell lung cancer in the subject, from archived subject samples, and the like. “Control” levels of expression of biomarkers of the invention may be available from publicly available databases. In addition, Universal Reference Total RNA (Clontech™) and Universal Human Reference RNA (Agilent Genomics™) and the like can be also used as “controls.” For example, quantitative polymerase reaction (qPCR) can be used to determine the level of expression of a biomarker, and an increase in the number of cycles needed to detect expression of a biomarker in a sample from a subject, relative to the number of cycles needed for detection using such a control, is indicative of a high or low level of expression of the biomarker.

Any biologic agent described herein, such as a therapeutic antibody or therapeutic protein described herein, encompasses any biosimilars thereof unless otherwise indicated. For example, a description of adalimumab would also encompass biosimilars e.g. adalimumab-atto, adalimumab-adbm, adalimumab-bwwd, and adalimumab-adaz.

As used herein, the term “subject” or “patient” refers to human and non-human animals, e.g., veterinary patients. The term “non-human animal” includes vertebrates, e.g., mammals, such as non-human primates, mice, rabbits, sheep, dog, cat, horse, cow, or other rodent, ovine, canine, feline, equine or bovine species. In one embodiment, the subject is a human.

The term “sample” as used herein refers to cells, tissues or fluids isolated from a subject, as well as cells, tissues or fluids present within a subject. The term “sample” includes any bodily fluid (e.g., blood, lymph, cystic fluid, expectorant, urine and fluids collected from a biopsy (e.g., needle lung biopsy)), tissue or a cell or collection of cells from a subject, as well as any component thereof, such as a fraction or extract. In one embodiment, the tissue or cell is removed from the subject. In another embodiment, the tissue or cell is present within the subject. Other samples include tears, plasma, serum, cerebrospinal fluid, feces, sputum and cell extracts. In one embodiment, the sample contains protein (e.g., proteins or peptides) from the subject.

In another embodiment, the sample contains RNA (e.g., mRNA molecules) from the subject or DNA (e.g., genomic DNA molecules) from the subject.

As used herein, the term “small cell lung cancer” refers generally to the uncontrolled growth of lung tissue and, more specifically, to a condition characterized by anomalous rapid proliferation of abnormal cells in one or both lungs of a subject. The abnormal cells often are referred to as malignant or “neoplastic cells,” which are transformed cells that can form a solid SCLC tumor. Tumors of small cell lung cancer include at least two different subtypes, only one of which is disclosed herein to be sensitive to treatment with pentamidine or a pharmaceutically acceptable salt thereof.

The term “tumor” refers to an abnormal mass or population of cells (i.e., two or more cells) that result from excessive or abnormal cell division, whether malignant or benign, and precancerous and cancerous cells. Malignant tumors are distinguished from benign growths or tumors in that, in addition to uncontrolled cellular proliferation, they can invade surrounding tissues and can metastasize. In small cell lung cancer, neoplastic cells may be identified in one or both lungs only and not in another tissue or organ, in one or both lungs and one or more adjacent tissues or organs, or in a lung and one or more nonadjacent tissues or organs to which the small cell lung cancer cells have metastasized.

The phrase “determining whether pentamindine, or a pharmaceutically acceptable salt thereof (e.g., pentamidine isethionate, pentamidine gluconate, or pentamidine mesylate), may be used to treat a subject having small cell lung cancer” refers to assessing the likelihood that treatment of a subject with pentamidine, or a pharmaceutically acceptable salt thereof (e.g., pentamidine isethionate, pentamidine gluconate, or pentamidine mesylate) will be effective (e.g., provide a therapeutic benefit to the subject as deemed a responder) or will not be effective in the subject (e.g., exclude the subject from pentamidine treatment as a non-responder). Assessment of the likelihood that treatment will or will not be effective typically can be performed before treatment with pentamidine, or a pharmaceutically acceptable salt thereof (e.g., pentamidine isethionate, pentamidine gluconate, or pentamidine mesylate), has begun or before treatment is resumed. Alternatively or in combination, assessment of the likelihood of efficacious treatment can be performed during treatment, for example, to determine whether treatment should be continued or discontinued. For example, such an assessment can be performed (a) by determining the level of expression of a biomarker, for example, a biomarker selected from the group of biomarkers listed in Table 1, Table 2, or Table 3 in a sample derived from said subject, wherein a high level of expression of biomarkers in Table 1 or Table 3 or a low level of expression of the biomarkers in Table 2 indicates that pentamidine, or a pharmaceutically acceptable salt thereof, may be used to treat said subject having small cell lung cancer, or (b) by assaying a sample derived from said subject to determine the level of expression in said sample of a biomarker, for example, a biomarker selected from the group of biomarkers listed in Table 1, Table 2 or Table 3 wherein a high level of expression of the biomarker listed in Table 1 or Table 3, or a low level of expression of the biomarker listed in Table 2 indicates that pentamidine, or a pharmaceutically acceptable salt thereof, may be used to treat said subject having lung cancer.

As used herein, the phrase “determining the sensitivity of a small cell lung tumor to treatment with pentamidine, or a pharmaceutically acceptable salt thereof” is intended to refer to assessing the susceptibility of a small cell lung tumor, e.g., small cell lung cancer cells, to treatment with pentamidine, or a pharmaceutically acceptable salt thereof (e.g., pentamidine isethionate, pentamidine gluconate, or pentamidine mesylate). Sensitivity of a tumor can include the ability of pentamidine, or a pharmaceutically acceptable salt thereof (e.g., pentamidine isethionate, pentamidine gluconate, or pentamidine mesylate), to kill tumor cells, to inhibit the spread and/or metastasis of tumor cells, and/or to inhibit the growth of tumor cells completely or partially (e.g., slow down the growth of tumor cells by at least 5%, 10%, 15%, 20%, 25%, 30%, 35%, 40%, 45%, 50%, 55%, 60%, 65%, 70%, 75%, 80%, 85%, 90%, or 95%). The assessment can be performed: (i) before treatment with pentamidine, or a pharmaceutically acceptable salt thereof (e.g., pentamidine isethionate, pentamidine gluconate, or pentamidine mesylate), is begun; (ii) before treatment is resumed in the subject; or (iii) during treatment, for example, to determine whether treatment should be continued or discontinued. For example, such a determination can be performed (a) by determining the level of expression of a biomarker, e.g., a biomarker selected from the group of biomarkers listed in Table 1, Table 2, or Table 3 in said tumor, wherein a high level of expression of the biomarker listed in Table 1 and optionally Table 3 in combination with a low level of expression of biomarker listed in Table 2 in said tumor indicates that said tumor is sensitive to treatment with pentamidine, or a pharmaceutically acceptable salt thereof, or (b) by determining the level of expression of a biomarker e.g., a biomarker selected from the group of biomarkers listed in Table 1 or Table 2 in said tumor, and identifying said tumor as being sensitive to treatment with pentamidine, or a pharmaceutically acceptable salt thereof, when said biomarker listed in Table 1 or Table 3 is expressed in said tumor at a high level and optionally in combination when said biomarker listed in Table 2 is expressed in said tumor at a low level.

As used herein, the term “pentamidine or a pharmaceutically acceptable salt thereof” refers to the art-recognized fully synthetic diamidine or a salt thereof as set forth in U.S. Pat. Nos. 2,277,861, 2,410,796, and 5,084,480, the entire contents of which are incorporated herein by reference and has the following structure:

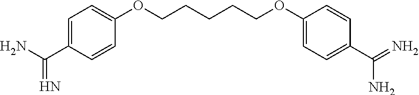 [SMILES: N=C(N)c1ccc(OCCCCCOc2ccc(C(=N)N)cc2)cc1]

### Pentamidine

Pentamidine can be generated using techniques as described in U.S. Pat. Nos. 2,277,861, 2,410,796, and 5,084,480, the entire contents of which are incorporated herein by reference. Pentamidine is also known as 4,4′-(Pentane-1,5-diylbis(oxy))dibenzimidamide; or 4-[5-(4-carbamimidoylphenoxy)pentoxy]benzenecarboximidamide and is identified by CAS number 100-33-4. Various pharmaceutically acceptable salts of pentamidine are also available as inhalant or injection in solution as powder. A “pharmaceutically acceptable salt” is a salt formed from an acid and a basic nitrogen group of pentamidine. Examples of such salts include acid addition salts and base addition salts, such as inorganic acid salts or organic acid salts (e.g., hydrochloric acid salt, sulfuric acid salt, citrate, hydrobromic acid salt, hydroiodic acid salt, nitric acid salt, bisulfate, phosphoric acid salt, super phosphoric acid salt, isonicotinic acid salt, acetic acid salt, lactic acid salt, salicylic acid salt, tartaric acid salt, pantothenic acid salt, ascorbic acid salt, succinic acid salt, maleic acid salt, fumaric acid salt, gluconic acid salt, saccharinic acid salt, formic acid salt, benzoic acid salt, glutaminic acid salt, methanesulfonic acid salt, ethanesulfonic acid salt, benzenesulfonic acid salt, p-toluenesulfonic acid salt, pamoic acid salt (pamoate)), as well as salts of aluminum, calcium, lithium, magnesium, calcium, sodium, zinc, and diethanolamine. It will be understood that reference to pentamidine or a pharmaceutically acceptable salt thereof, includes pharmaceutically acceptable salts of pentamidine. Examples of such pharmaceutically acceptable salts include, but are not limited to, pentamidine isethionate, pentamidine gluconate, and pentamidine mesylate. For example, pentamidine isethionate is identified by CAS number 140-64-7; pentamidine gluconate is identified by CAS number 123245-08-9; and pentamidine mesylate is identified by CAS number 6823-79-6. Pentamidine isethionate, for example, is commercially available as a lyophilized white crystalline powder for reconstitution with sterile water or 5% Dextrose for injection.

### Methods

I. Prediction of Responsiveness to Pentamidine or a Pharmaceutically Acceptable Salt Thereof in a Subject with Small Cell Lung Cancer

The present invention provides methods for determining whether pentamidine, or a pharmaceutically acceptable salt thereof (e.g., pentamidine isethionate, pentamidine gluconate, or pentamidine mesylate), can be used to treat a subject having small cell lung cancer, methods for predicting whether pentamidine, or a pharmaceutically acceptable salt thereof (e.g., pentamidine isethionate, pentamidine gluconate, or pentamidine mesylate), may be used to treat a subject having small cell lung cancer, methods for determining the sensitivity of a small cell lung tumor to treatment with pentamidine, or a pharmaceutically acceptable salt thereof (e.g., pentamidine isethionate, pentamidine gluconate, or pentamidine mesylate), and methods of treating a subject having small cell lung cancer by diagnosing subtype of small cell lung cancer and administering the subject with an effective amount of pentamidine or a pharmaceutically acceptable salt thereof (e.g., pentamidine isethionate, pentamidine gluconate, or pentamidine mesylate) when it is determined that the subject indeed suffers from a subtype of small cell lung cancer that may be treated with pentamidine or a pharmaceutically acceptable salt thereof (e.g., pentamidine isethionate, pentamidine gluconate, or pentamidine mesylate).

Accordingly, in some aspects, provided herein are methods for predicting the responsiveness of a subject with small cell lung cancer to treatment with pentamidine, or a pharmaceutically acceptable salt thereof, the method comprising determining the level of expression of at least one biomarker selected from the group consisting of INSM1, GADD45G, and C1QL1 by the small cell lung cancer, wherein the subject is predicted to be responsive to small cell lung cancer treatment with a therapeutically effective amount of pentamidine or a pharmaceutically acceptable salt thereof if the small cell lung cancer expresses a high level of the at least one biomarker selected from the group consisting of INSM1, GADD45G, and C1QL1. In some aspects, provided herein are methods for identifying a subject suitable for small cell lung cancer treatment, the method comprising determining the level of expression of at least one biomarker selected from the group consisting of INSM1, GADD45G, and C1QL1 by the small cell lung cancer, wherein the subject is identified as suitable for small cell lung cancer treatment with a therapeutically effective amount of pentamidine or a pharmaceutically acceptable salt thereof if the small cell lung cancer expresses a high level of the at least one biomarker selected from the group consisting of INSM1, GADD45G, and C1QL1.

In some aspects, provided herein are methods for monitoring the responsiveness of a subject with small cell lung cancer to treatment with pentamidine, or a pharmaceutically acceptable salt thereof, the method comprising determining the level of expression of at least one biomarker selected from the group consisting of INSM1, GADD45G, and C1QL1 by the small cell lung cancer, administering a therapeutically effective amount of pentamidine or a pharmaceutically acceptable salt thereof, to said subject, and determining the level of expression of at least one biomarker selected from the group consisting of INSM1, GADD45G, and C1QL1 by the small cell lung cancer after the administering, wherein the subject is responsive to small cell lung cancer treatment if the small cell lung cancer expresses a lower level of the at least one biomarker selected from the group consisting of INSM1, GADD45G, and C1QL1 after the administering compared to prior to the administering.

Generally, in some embodiments, the methods involve determining the level of expression of at least one biomarker as set forth in Table 1, Table 2 and/or Table 3 in a sample derived from the subject, wherein a high level of expression of the biomarker listed in Table 1 is an indication that pentamidine or a pharmaceutically acceptable salt thereof may be used to treat small cell lung cancer and/or that the small cell lung tumor is sensitive to treatment with pentamidine or a pharmaceutically acceptable salt thereof (e.g., pentamidine isethionate, pentamidine gluconate, or pentamidine mesylate). Also provided herein are methods for administering to a subject (e.g., human patient), a pharmaceutical composition comprising pentamidine or a pharmaceutically acceptable salt thereof to treat a subtype of small cell lung cancer associated with a certain biomarker or a certain set of biomarker, for example, one or more biomarkers listed in Table 1, Table 2 and/or Table 3 based on their biomarker expression profile.

In some aspects, the expression level of at least one biomarker selected from the group of biomarkers set forth in Table 1 and Table 2 is assessed, which, as explained herein, can comprise determining the level of expression of one or more of these genes (e.g., INSM1, GADD45G, STAT6, SMAD3, C1QL1, and RNF43) in a tumor sample derived from subject's small cell lung cancer, using various approaches, such as determining the level of RNA expressed from a gene, including an mRNA exemplified in Table 1, Table 2 and/or other transcripts from the gene, or a protein product(s) of any of the foregoing to establish biomarker expression signature. In one embodiment, said at least one biomarker is INSM1. In combination with an INSM1 biomarker expression signature, for example, a predictive gene expression profile can be assessed based on a biomarker expression signature of GADD45G, STAT6, SMAD3, C1QL1, RNF43 or any combination thereof. In one embodiment, a predictive gene expression signature to be used in determining susceptibility or sensitivity of a subtype of small cell lung cancer to treatment with pentamidine or a pharmaceutically acceptable salt thereof (e.g., pentamidine isethionate, pentamidine gluconate, or pentamidine mesylate) is biomarker expression of INSM1 in conjunction with biomarker expression signature selected from the group of GADD45G, STAT6, SMAD3, C1QL1, RNF43 or any combination thereof.

The level of expression of the biomarker in a sample can be determined using methods that involve the use of nucleic acid amplification and/or reverse transcriptase (to prepare cDNA) of for example mRNA in the sample, for example, by RT-PCR (the experimental embodiment set forth in Mullis, 1987, U.S. Pat. No. 4,683,202), ligase chain reaction (Barany (1991) Proc. Natl. Acad. Sci. USA 88:189-193), self-sustained sequence replication (Guatelli et al. (1990) Proc. Natl. Acad. Sci. USA 87:1874-1878), transcriptional amplification system (Kwoh et al. (1989) Proc. Natl. Acad. Sci. USA 86:1173-1177), Q-Beta Replicase (Lizardi et al. (1988) Bio/Technology 6:1197), rolling circle replication (Lizardi et al., U.S. Pat. No. 5,854,033) or any other nucleic acid amplification method, followed by the detection of the amplified molecules. These approaches are especially useful for the detection of nucleic acid molecules if such molecules are present in very low numbers. In particular aspects of the invention, the level of expression of the biomarker is determined by quantitative fluorogenic RT-PCR (e.g., the TaqMan™ System). Alternatively or in combination, other RNA quantification methods including hybridization based microarrays and RNA-seq can be employed. Such methods typically utilize pairs of oligonucleotide primers that are specific for the biomarker. Methods for designing oligonucleotide primers specific for a known sequence are well known in the art. The expression levels of biomarker mRNA can be monitored using a membrane blot (such as used in hybridization analysis such as Northern, Southern blot, and the like), or microwells, sample tubes, gels, beads or fibers (or any solid support comprising bound nucleic acids). The determination of biomarker expression level may also comprise using nucleic acid probes in solution.

In one embodiment of the invention, microarrays are used to detect the level of expression of a biomarker. Microarrays are particularly well suited for this purpose because of the reproducibility between different experiments. DNA microarrays provide one method for the simultaneous measurement of the expression levels of large numbers of genes. Each array consists of a reproducible pattern of capture probes attached to a solid support. Labeled RNA or DNA is hybridized to complementary probes on the array and then detected by laser scanning. Hybridization intensities for each probe on the array are determined and converted to a quantitative value representing relative gene expression levels. See, e.g., U.S. Pat. Nos. 6,040,138, 5,800,992 and 6,020,135, 6,033,860, and 6,344,316, the contents of which as they relate to these assays are incorporated herein by reference. High-density oligonucleotide arrays are particularly useful for determining the gene expression profile for a large number of RNA's in a sample.

Expression of a biomarker can also be assessed at the protein level, using a detection reagent that detects the protein product encoded by the mRNA of the biomarker, directly or indirectly. For example, if an antibody reagent is available that binds specifically to a biomarker protein product to be detected, then such an antibody reagent can be used to detect the expression of the biomarker in a sample from the subject, using techniques, such as immunohistochemistry, ELISA, FACS analysis, and the like. Other known methods for detecting the biomarker at the protein level include methods such as electrophoresis, capillary electrophoresis, high performance liquid chromatography (HPLC), thin layer chromatography (TLC), hyperdiffusion chromatography, and the like, or various immunological methods such as fluid or gel precipitation reactions, immunodiffusion (single or double), immunoelectrophoresis, radioimmunoassay (MA), enzyme-linked immunosorbent assays (ELISAs), immunofluorescent assays, and Western blotting. Proteins from samples can be isolated using a variety of techniques, including those well known to those of skill in the art.

It is generally preferable to immobilize either the antibody or proteins on a solid support for Western blots and immunofluorescence techniques. Suitable solid phase supports or carriers include any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. One skilled in the art will know many other suitable carriers for binding antibody or antigen, and will be able to adapt such support for use with the present invention. For example, protein isolated from cells can be run on a polyacrylamide gel electrophoresis and immobilized onto a solid phase support such as nitrocellulose. The support can then be washed with suitable buffers followed by treatment with the detectably labeled antibody. The solid phase support can then be washed with the buffer a second time to remove unbound antibody. The amount of bound label on the solid support can then be detected by conventional means. Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purificaction, Academic Press, Inc. N.Y.).

Antibodies used in immunoassays to determine the level of expression of the biomarker, may be labeled with a detectable label. The term “labeled,” with regard to the probe or antibody, is intended to encompass direct labeling of the probe or antibody by incorporation of a label (e.g., a radioactive atom), coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently labeled secondary antibody and end-labeling of a DNA probe with biotin such that it can be detected with fluorescently labeled streptavidin.

In one embodiment, the antibody is labeled, for example, a radio-labeled, chromophore-labeled, fluorophore-labeled, or enzyme-labeled antibody. In another embodiment, an antibody derivative (e.g., an antibody conjugated with a substrate or with the protein or ligand of a protein-ligand pair (e.g., biotin-streptavidin), or an antibody fragment (e.g. a single-chain antibody, or an isolated antibody hypervariable domain) which binds specifically with the biomarker is used. In one embodiment, proteomic methods, for example, mass spectrometry, are used. Mass spectrometry is an analytical technique that consists of ionizing chemical compounds to generate charged molecules (or fragments thereof) and measuring their mass-to-charge ratios. In a typical mass spectrometry procedure, a sample is obtained from a subject, loaded onto the mass spectrometry, and its components (e.g., the biomarker) are ionized by different methods (e.g., by impacting them with an electron beam), resulting in the formation of charged particles (ions). The mass-to-charge ratio of the particles is then calculated from the motion of the ions as they transit through electromagnetic fields.

For example, matrix-associated laser desorption/ionization time-of-flight mass spectrometry (MALDI-TOF MS) or surface-enhanced laser desorption/ionization time-of-flight mass spectrometry (SELDI-TOF MS) which involves the application of a biological sample, such as serum, to a protein-binding chip (Li, J., et al. (2002) Clin Chem 48:1296; Laronga, C., et al. (2003) Dis Biomarkers 19:229; Adam, B. L., et al. (2002) Cancer Res 62:3609; Tolson, J., et al. (2004) Lab Invest 84:845; Xiao, Z., et al. (2001) Cancer Res 61:6029) can be used to determine the expression level of a biomarker at the protein level.

Furthermore, in vivo techniques for determination of the expression level of the biomarker include introducing into a subject a labeled antibody directed against the biomarker, which binds to and transforms the biomarker into a detectable molecule. As discussed above, the presence, level, or even location of the detectable biomarker in a subject may be detected by standard imaging techniques.

The presence or level of the protein can be detected using an antibody or antigen binding fragment thereof, which specifically binds to the protein. In particular embodiments, the antibody or antigen binding fragment thereof is selected from the group consisting of a murine antibody, a human antibody, a humanized antibody, a bispecific antibody, a chimeric antibody, a Fab, Fab′, F(ab′)₂, ScFv, nanobody, affibody, and a domain antibody, or an antigen binding fragment of any of the foregoing. In certain embodiments, the level of expression of the biomarker is determined by using a technique selected from the group consisting of a western blot analysis, an immunoassay, immunofluorimetry, a radioimmunoassay, immunoprecipitation, immunodiffusion, equilibrium dialysis, ELISA assay, electrochemiluminescence (ECL), immunopolymerase chain reaction and combinations or sub-combinations thereof. In particular embodiments, the immunoassay is a solution-based immunoassay selected from the group consisting of fluorogenic chemiluminescence, electrochemiluminescence, chemiluminescence, fluorescence polarization, and time-resolved fluorescence. In other embodiments, the immunoassay is a sandwich immunoassay selected from the group consisting of chemiluminescence, electrochemiluminescence, and fluorogenic chemiluminescence. Other assays which rely on agents capable of detecting the protein, such as those relying upon a suitable binding partner or enzymatic activity, can also be used (e.g., use of a ligand to detect a receptor molecule).

Samples can be obtained from a subject by any suitable method, and may optionally have undergone further processing step(s) (e.g., freezing, fractionation, fixation, guanidine treatment, etc.). Any suitable sample derived from a subject can be used, such as any tissue (e.g., biopsy), cell, or fluid, as well as any component thereof, such as a fraction or extract. In various embodiments, the sample is a fluid obtained from the subject, or a component of such a fluid. For example, the fluid can be blood, plasma, serum, sputum, lymph, cystic fluid, urine, or fluid collected from a biopsy (e.g., needle biopsy). In other embodiments, the sample is a tissue or component thereof obtained from the subject. For example, the tissue can be a tissue obtained from a biopsy from a lung tissue, lymphatic tissue or blood samples. In a particular embodiment, the tissue is a small cell lung cancer tissue, or a component thereof (e.g., cells collected from the lung tissue). In a particular embodiment, the component of the lung tissue are small cell lung cancer tissue cells. In another embodiment, the component of the small cell lung tissue is the circulating small cell lung tumor cell.

In certain embodiments, the level of expression of the biomarker can be determined by detecting miRNA. Specifically, mRNA expression can be assessed indirectly by assessing levels of miRNA, where a low level of a miRNA which controls the expression of an mRNA is indicative of a high level of expression of the mRNA encoding the biomarker. Conversely, an elevated level of a miRNA which controls the expression of an mRNA is indicative of a low level of expression of the mRNA encoding the biomarker.

In general, where a difference in the level of expression of a biomarker and the control is to be detected, it is preferable that the difference between the level of expression of the biomarker in a sample from a subject having small cell lung cancer and being treated with pentamidine, or a pharmaceutically acceptable salt thereof (e.g., pentamidine isethionate, pentamidine gluconate, or pentamidine mesylate), or being considered for treatment with pentamidine, or a pharmaceutically acceptable salt thereof (e.g., pentamidine isethionate, pentamidine gluconate, or pentamidine mesylate), and the amount of the biomarker in a control sample, is as great as possible. Although this difference can be as small as the limit of detection of the method for determining the level of expression, it is preferred that the difference be greater than the limit of detection of the method or greater than the standard error of the assessment method, and preferably a difference of at least about 2-, about 3-, about 4-, about 5-, about 6-, about 7-, about 8-, about 9-, about 10-, about 15-, about 20-, about 25-, about 100-, about 500-, 1000-fold greater than the standard error of the assessment method. A small cell lung cancer sample is a sample, as described herein, that is capable of yielding information regarding the expression level of a biomarker by small cell lung cancer cells of the subject.

Any suitable sample obtained from a subject having small cell lung cancer can be used to assess the level of expression, including a high expression or a low/lack of expression, of the biomarker(s), for example, a biomarker provided in Table 1 and Table 2, and optionally Table 3. For example, the sample may be any fluid or component thereof, such as a fraction or extract, e.g., blood, plasma, lymph, cystic fluid, urine, samples from needle lung, or fluids collected from a biopsy (e.g., lung biopsy), obtained from the subject. In a typical situation, the fluid may be blood, or a component thereof, obtained from the subject, including whole blood or components thereof, including, plasma, serum, and blood cells, such as red blood cells, white blood cells and platelets. The sample may also be any tissue or fragment or component thereof, for example, lung tissue, connective tissue, lymph tissue or muscle tissue obtained from the subject. Techniques or methods for obtaining samples from a subject are well known in the art and include, for example, obtaining samples by a mouth swab or a mouth wash; drawing blood; or obtaining a biopsy. Isolating components of fluid or tissue samples (e.g., cells or RNA or DNA) may be accomplished using a variety of techniques.

The sample from the cancer may be obtained by biopsy of the patient's cancer. In certain embodiments, more than one sample from the patient's tumor is obtained in order to acquire a representative sample of cells for further study. For example, a patient with small cell lung cancer may have a needle biopsy to obtain a sample of small cell lung cancer cells. Several biopsies of the tumor may be used to obtain a sample of cancer cells. In other embodiments, the sample may be obtained from surgical excision of the small cell lung tumor. In this case, one or more samples may be taken from the excised tumor for analysis using the methods of the invention.

After the sample is obtained, it may be further processed. The cancer cells may be cultured, washed, or otherwise selected to remove normal tissue. The cells may be trypsinized to remove the cells from the tumor sample. The cells may be sorted by fluorescence activated cell sorting (FACS) and cultured to obtain a greater number of cells for study. In certain instances, the cells may be immortalized. For some applications, the cells may be frozen or the cells may be embedded in paraffin.

II. Treatment with Pentamidine or a Pharmaceutically Acceptable Salt Thereof

In some embodiments, the methods provided herein include: (1) identification of a subject having small cell lung cancer in which at least one biomarker selected from the group of biomarkers listed in Table 1, and optionally at least one biomarker select\ed from the group of biomarkers listed Table 2 or Table 3; and (ii) administration of a therapeutically effective amount of pentamidine or a pharmaceutically acceptable salt thereof (e.g., pentamidine isethionate, pentamidine gluconate, or pentamidine mesylate) to the subject. For examples, the level of expression of one or more, e.g., at least 1, 2, 3, 4, 5, or 6 biomarkers of Table 1 or Table 2 is determined. If expression level(s) of at least one biomarker(s) listed in Table 1 is determined to be high and at least one biomarker in Table 2 is determined to be low as compared to the respective control (e.g., normal lung tissue or a different subtype of SCLC), treatment with pentamidine, or a pharmaceutically acceptable salt thereof (e.g., pentamidine isethionate, pentamidine gluconate, or pentamidine mesylate), is likely to be efficacious. Preferably, said at least one biomarker from Table 1 is INSM1.

Given the observation that the expression levels of certain biomarkers, for example, those set forth in Table 1 and/or Table 2, in a subject having small cell lung cancer influences the responsiveness of the subject to pentamidine, or a pharmaceutically acceptable salt thereof (e.g., pentamidine isethionate, pentamidine gluconate, or pentamidine mesylate), a skilled artisan can select an appropriate treatment regimen for the subject based on the expression levels of the biomarkers in the subject.

Accordingly, the present invention provides methods for treating a subject having small cell lung cancer by (i) assaying a small cell lung cancer sample derived from the subject to determine the level of expression in said sample of at least one biomarker selected from the group of biomarkers listed in Table 1 and optionally at least one additional biomarker from Table 2 or Table 3; (ii) detecting a high level of expression of said at least one biomarker in Table 1 and optionally in Table 3 in said sample relative to a normal control (e.g., normal lung tissue); and (iii) optionally detecting a low level of expression of said at least one biomarker in Table 2 in said sample relative to a normal control (e.g., normal lung tissue); and (iv) administering a therapeutically effective amount of pentamidine, or a pharmaceutically acceptable salt thereof (e.g., pentamidine isethionate, pentamidine gluconate, or pentamidine mesylate) to the subject when a high level of expression of the at least one biomarker from Table 1 and Table 3; and optionally a low level of expression of the at least one biomarker from Table 2 are detected in the sample. Preferably, said at least one biomarker from Table 1 is INSM1.

Also provided herein are methods of treating a subject suffering from small cell lung cancer expressing a high level of at least one biomarker selected from the group consisting of INSM1, GADD45G, and C1QL1, the method comprising administering a therapeutically effective amount of pentamidine or a pharmaceutically acceptable salt thereof, to the subject. In some embodiments, the small cell lung cancer further expresses a low level of a low level of at least one biomarker selected from the group consisting of STAT6, SMAD3, and RNF43 and/or a high level of one or more biomarkers listed in Table 3.

Also provided herein are methods of treating a subject suffering from small cell lung cancer, the method comprising administering a therapeutically effective amount of pentamidine or a pharmaceutically acceptable salt thereof, to the subject, wherein expression of a high level of at least one biomarker selected from the group consisting of INSM1, GADD45G, and C1QL1 by the small cell lung cancer is used as a basis for selecting the subject to receive treatment.

Also provided herein are methods of treating a subject suffering from small cell lung cancer, the method comprising a) determining the level of expression by the small cell lung cancer of at least one biomarker selected from the group consisting of INSM1, GADD45G, and C1QL1; and b) administering a therapeutically effective amount of pentamidine or a pharmaceutically acceptable salt thereof, to said subject, wherein the small cell lung cancer expresses a high level of the at least one biomarker selected from the group consisting of INSM1, GADD45G, and C1QL1. In some embodiments, the method further comprises determining the level of expression by the small cell lung cancer of at least one biomarker selected from the group consisting of INSM1, GADD45G, and C1QL1 after the administering. In some embodiments, the method further comprises administering a therapeutic agent that does not comprise pentamidine or a pharmaceutically acceptable salt thereof. In some embodiments, the method further comprises administering a therapeutic agent that does not comprise pentamidine or a pharmaceutically acceptable salt thereof if the level of expression of the at least one biomarker selected from the group consisting of INSM1, GADD45G, and C1QL1 is not lower after the administering compared with prior to the administering. In some embodiments, the method further comprises administering a therapeutically effective amount of etoposide, irinotecan, cisplatin, carboplatin, atezolizumab, ipilimumab, nivolumab, pembrolizumab, or combinations thereof. In some embodiments, the method further comprises administering etopside or irinotecan; and cisplatin or carboplatin. In some embodiments, the method further comprises administering etopside or irinotecan; cisplatin or carboplatin; and atezolizumab, ipilimumab, nivolumab, or pembrolizumab.

Also provided herein are methods of treating a subject suffering from small cell lung cancer, the method comprising a) determining the level of expression by the small cell lung cancer of at least one biomarker selected from the group consisting of INSM1, GADD45G, and C1QL1; and b) (i) administering a therapeutically effective amount of pentamidine or a pharmaceutically acceptable salt thereof, to said subject, wherein the small cell lung cancer expresses a high level of the at least one biomarker selected from the group consisting of INSM1, GADD45G, and C1QL1; or (ii) administering a therapeutically effective amount of a therapeutic agent that does not comprise pentamidine or a pharmaceutically acceptable salt thereof. In some embodiments, the method comprises a) determining the level of expression by the small cell lung cancer of at least one biomarker selected from the group consisting of INSM1, GADD45G, and C1QL1; and b) (i) administering a therapeutically effective amount of pentamidine or a pharmaceutically acceptable salt thereof, to said subject, wherein the small cell lung cancer expresses a high level of the at least one biomarker selected from the group consisting of INSM1, GADD45G, and C1QL1; or (ii) administering a therapeutically effective amount of etoposide, irinotecan, cisplatin, carboplatin, atezolizumab, ipilimumab, nivolumab, pembrolizumab, or combinations thereof. In some embodiments, the method comprises (ii) administering etopside or irinotecan; and cisplatin or carboplatin. In some embodiments, the method comprises (ii) administering etopside or irinotecan; cisplatin or carboplatin; and atezolizumab, ipilimumab, nivolumab, or pembrolizumab.

In one embodiment, when high levels of expression of one or more, for example, at least 1, 2, or 3 biomarkers identified in Table 1 is determined (e.g., INSM1), treatment with pentamidine, or a pharmaceutically acceptable salt thereof (e.g., pentamidine isethionate, pentamidine gluconate, or pentamidine mesylate), is likely to be efficacious.

However, it is not necessary that all of the biomarkers assayed have a high level of expression as compared to the respective control. For example, while certain biomarkers may be present at high levels of expression, treatment with pentamidine, or a pharmaceutically acceptable salt thereof (e.g., pentamidine isethionate, pentamidine gluconate, or pentamidine mesylate), may be indicated when, for example, a normal or low level of expression is present for at least 1 or 2 biomarkers listed in Table 2. In a preferred embodiment, when a high level of expression of INSM1 and one or more additional biomarker in Table 1 in conjunction with a low level of expression of one or more biomarkers listed in Table 2 are found in a sample derived from a subject having small cell lung cancer, the subject may be treated with pentamidine, or a pharmaceutically acceptable salt of thereof (e.g., pentamidine isethionate, pentamidine gluconate, or pentamidine mesylate) with a high degree of certainty that treatment with pentamidine or a pharmaceutically acceptable salt of pentamidine will be efficacious.

### Biomarkers

The present invention relates to treatment of small cell lung cancer predicted to be responsive to treatment with pentamidine or a pharmaceutically acceptable salt thereof. The biomarkers described in Tables 1, 2, and 3 exhibit two properties: (1) expression profiles strongly correlates with a subtype of small cell lung cancer predicted to be susceptible or sensitive to treatment with pentamidine or a pharmaceutically acceptable salt thereof and (2) the expression profile of these biomarkers is responsive to the treatment with pentamidine or a pharmaceutically acceptable salt of pentamidine, meaning that the biomarker expression profile representing a subtype of diseased SCLC tumors is highly likely to be reversed upon the treatment with pentamidine to resemble the expression pattern of normal controls, e.g., normal lung cells. Accordingly, the present invention involves methods for determining whether pentamidine or a pharmaceutically acceptable salt thereof, may be used to treat a subject having small cell lung cancer or for determining the sensitivity of small cell lung cancer to treatment with pentamidine or a pharmaceutically acceptable salt thereof.

Prediction for potential responders to pentamidine with small cell lung cancer can be made by measuring gene expression levels of biomarkers identified herein. As noted herein, the invention is based on a discovery that a set of biomarkers is perturbed in a distinctive way that is consistent with a subtype of small cell lung cancer. Various biomarkers to be used in the methods are summarized Table 1, Table 2 and Table 3. Tables 1, 2 and 3 provide gene abbreviations, and accession numbers for transcripts from which encoding nucleotide gene sequences can be identified. For example, gene INSM1 has RNA accession number NM 002196 that encodes insulinoma-associated protein 1. It is found that expression of INSM1 and other biomarkers described in this disclosure are significantly associated with a subtype of small cell lung cancer and also responses to treatment with pentamidine or a pharmaceutically acceptable salt thereof. Reference to a gene (e.g., INSM1) is intended to encompass naturally occurring or endogenous versions of the gene, including wild-type, allelic or polymorphic variants or mutants of the gene, which can be found in a subject and/or tumor from a subject. In some embodiments, the sequence of the biomarker gene is at least about 80%, at least about 85%, at least about 90%, at least about 95%, at least about 97%, and at least about 99% identical to a sequence identified in Table 1 by NM accession numbers. For example, sequence identity can be determined by comparing sequences using NCBI BLAST (e.g., Megablast with default parameters). Each of the accession numbers identified in Table 1 or Table 2 and their corresponding sequences are hereby incorporated herein by reference.

In various embodiments, the expression levels (e.g., transcriptome analysis using e.g., microarray or RNA-seq; or protein level analysis using ELISA, ECL, or Western blotting) of at least 1, 2, 3, 4, 5 and 6 biomarkers selected from the group of biomarker genes listed in Table 1 or Table 2 can be determined. A predictive gene expression signature comprising at least one biomarker of Table 1 can be also used. For example, the INSM1 gene expression signature predicts positive responses to treatment with pentamidine. A predictive gene expression signature comprising INSM1 and a sub-combination of two or more biomarkers selected from the group of biomarkers listed in Table 1, Table 2 and Table 3 can be used.

In various embodiments, the level of expression of at least 2, at least 3 or at least 4 biomarkers selected from the group of biomarkers listed in Table 1 and 2 is determined. For examples, the predictive gene expression signature may include INSM1 and at least one biomarker selected from the group consisting of GADD45G, STAT6, SMAD3, C1QL1 and RNF43. In some embodiments, the predictive gene expression signature may include at least three biomarkers comprising INSM1 and one or more additional biomarkers selected from the group consisting of GADD45G, STAT6, SMAD3, C1QL1, and RNF43 (see Table 4 for the statistical precision and recall rates for predicting the SCLC subtype that is susceptible to pentamidine or a pharmaceutically acceptable salt thereof).

In some embodiments, the predictive gene expression signature may include at least three biomarkers comprising INSM1 and two or more additional biomarkers selected from the group consisting of GADD45G, STAT6, SMAD3, C1QL1, and RNF43.

In some embodiments, the predictive gene expression signature may include at least four biomarkers comprising INSM1 and three or more additional biomarkers selected from the group consisting of GADD45G, STAT6, SMAD3, C1QL1, and RNF43.

In some embodiments, the predictive gene expression signature may include at least five biomarkers comprising INSM1 and four or more additional biomarkers selected from the group consisting of GADD45G, STAT6, SMAD3, C1QL1, and RNF43.

In one embodiment, the predictive gene expression signature includes at least six biomarkers comprising INSM1, GADD45G, STAT6, SMAD3, C1QL1, and RNF43.

In some embodiments, the small cell lung cancer expresses a high level of INSM1. In some embodiments, the small cell lung cancer expresses a high level of GADD45G. In some embodiments, the small cell lung cancer expresses a high level of C1QL1. In some embodiments, the small cell lung cancer expresses a high level of INSM1 and GADD45G. In some embodiments, the small cell lung cancer expresses a high level of INSM1 and C1QL1. In some embodiments, the small cell lung cancer expresses a high level of GADD45G and C1QL1. In some embodiments, the small cell lung cancer expresses a high level of INSM1, GADD45G, and C1QL1.

In some embodiments, the small cell lung cancer expresses a low level of STAT6. In some embodiments, the small cell lung cancer expresses a low level of SMAD3. In some embodiments, the small cell lung cancer expresses a low level of RNF43. In some embodiments, the small cell lung cancer expresses a low level of STAT6 and SMAD3. In some embodiments, the small cell lung cancer expresses a low level of STAT6 and RNF43. In some embodiments, the small cell lung cancer expresses a low level of SMAD3 and RNF43.In some embodiments, the small cell lung cancer expresses a low level of STAT6, SMAD3, and RNF43.

In some embodiments, the small cell lung cancer expresses a high level of INSM1 and C1QL1, and a low level of RNF43. In some embodiments, the small cell lung cancer expresses a high level of INSM1, and C1QL1, and a low level of SMAD3. In some embodiments, the small cell lung cancer expresses a high level of INSM1 and C1QL1, and a low level of SMAD3 and RNF43. In some embodiments, the small cell lung cancer expresses a high level of INSM1 and C1QL1, and a low level of STAT6. In some embodiments, the small cell lung cancer expresses a high level of INSM1 and C1QL1, and a low level of STAT6 and RNF43. In some embodiments, the small cell lung cancer expresses a high level of INSM1 and C1QL1, and a low level of STAT6 and SMAD3. In some embodiments, the small cell lung cancer expresses a high level of INSM1 and C1QL1, and a low level of STAT6, SMAD3, and RNF43. In some embodiments, the small cell lung cancer expresses a high level of INSM1, GADD45G, and C1QL1, and a low level of RNF43. In some embodiments, the small cell lung cancer expresses a high level of INSM1, GADD45G, and C1QL1, and a low level of SMAD3. In some embodiments, the small cell lung cancer expresses a high level of INSM1, GADD45G, and C1QL1, and a low level of SMAD3, RNF43. In some embodiments, the small cell lung cancer expresses a high level of INSM1, GADD45G, and C1QL1, and a low level of STAT6 and RNF43. In some embodiments, the small cell lung cancer expresses a high level of INSM1, GADD45G, and C1QL1, and a low level of STAT6 and SMAD3. In some embodiments, the small cell lung cancer expresses a high level of INSM1, GADD45G, and C1QL1, and a low level of STAT6, SMAD3, and RNF43. In some embodiments, the small cell lung cancer expresses a high level of INSM1, GADD45G, and and a low level of RNF43. In some embodiments, the small cell lung cancer expresses a high level of INSM1 and GADD45G, and a low level of SMAD3 and RNF43. In some embodiments, the small cell lung cancer expresses a high level of INSM1 and GADD45G, and a low level of STAT6 and RNF43. In some embodiments, the small cell lung cancer expresses a high level of INSM1 and GADD45G, and a low level of STAT6 and SMAD3. In some embodiments, the small cell lung cancer expresses a high level of INSM1 and GADD45G, and a low level of STAT6, SMAD3, and RNF43. In some embodiments, the small cell lung cancer expresses a high level of INSM1, and a low level of SMAD3. In some embodiments, the small cell lung cancer expresses a high level of INSM1, and a low level of SMAD3 and RNF43. In some embodiments, the small cell lung cancer expresses a high level of INSM1, and a low level of STAT6. In some embodiments, the small cell lung cancer expresses a high level of INSM1, and a low level of STAT6 and RNF43. In some embodiments, the small cell lung cancer expresses a high level of INSM1, and a low level of STAT6 and SMAD3. In some embodiments, the small cell lung cancer expresses a high level of INSM1, and a low level of STAT6, SMAD3, and RNF43.

In some embodiments, the small cell lung cancer further expresses a high level of one or more of the biomarkers listed in Table 3, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 biomarkers listed in Table 3.

In some embodiments, the method comprises determining the expression level of INSM1. In some embodiments, the method comprises determining the expression level of GADD45G. In some embodiments, the method comprises determining the expression level of C1QL1. In some embodiments, the method comprises determining the expression level of INSM1 and GADD45G. In some embodiments, the method comprises determining the expression level of INSM1 and C1QL1. In some embodiments, the method comprises determining the expression level of GADD45G and C1QL1. In some embodiments, the method comprises determining the expression level of INSM1, GADD45G, and C1QL1.

In some embodiments, the method comprises determining the expression level of STAT6. In some embodiments, the method comprises determining the expression level of SMAD3. In some embodiments, the method comprises determining the expression level of RNF43. In some embodiments, the method comprises determining the expression level of STAT6 and SMAD3. In some embodiments, the method comprises determining the expression level of STAT6 and RNF43. In some embodiments, the method comprises determining the expression level of SMAD3 and RNF43. In some embodiments, the method comprises determining the expression level of STAT6, SMAD3, and RNF43.

In some embodiments, the method comprises determining the expression level of INSM1, C1QL1, and RNF43. In some embodiments, the method comprises determining the expression level of INSM1, C1QL1, and SMAD3. In some embodiments, the method comprises determining the expression level of INSM1, C1QL1, SMAD3, and RNF43. In some embodiments, the method comprises determining the expression level of INSM1, C1QL1, and STAT6. In some embodiments, the method comprises determining the expression level of INSM1, C1QL1, STAT6, and RNF43. In some embodiments, the method comprises determining the expression level of INSM1, C1QL1, STAT6, and SMAD3. In some embodiments, the method comprises determining the expression level of INSM1, C1QL1, STAT6, SMAD3, and RNF43. In some embodiments, the method comprises determining the expression level of INSM1, GADD45G, C1QL1, and RNF43. In some embodiments, the method comprises determining the expression level of INSM1, GADD45G, C1QL1, and SMAD3. In some embodiments, the method comprises determining the expression level of INSM1, GADD45G, C1QL1, SMAD3, and RNF43. In some embodiments, the method comprises determining the expression level of INSM1, GADD45G, C1QL1, STAT6, and RNF43. In some embodiments, the method comprises determining the expression level of INSM1, GADD45G, C1QL1, STAT6, and SMAD3. In some embodiments, the method comprises determining the expression level of INSM1, GADD45G, C1QL1, STAT6, SMAD3, and RNF43. In some embodiments, the method comprises determining the expression level of INSM1, GADD45G, and RNF43. In some embodiments, the method comprises determining the expression level of INSM1, GADD45G, SMAD3, and RNF43. In some embodiments, the method comprises determining the expression level of INSM1, GADD45G, STAT6, and RNF43. In some embodiments, the method comprises determining the expression level of INSM1, GADD45G, STAT6, and SMAD3. In some embodiments, the method comprises determining the expression level of INSM1, GADD45G, STAT6, SMAD3, and RNF43. In some embodiments, the method comprises determining the expression level of INSM1 and SMAD3. In some embodiments, the method comprises determining the expression level of INSM1, SMAD3, and RNF43. In some embodiments, the method comprises determining the expression level of INSM1 and STAT6. In some embodiments, the method comprises determining the expression level of INSM1, STAT6, and RNF43. In some embodiments, the method comprises determining the expression level of INSM1, STAT6, and SMAD3. In some embodiments, the method comprises determining the expression level of INSM1, STAT6, SMAD3, and RNF43.

In some embodiments, the method further comprises determining the expression level of one or more of the biomarkers listed in Table 3, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 biomarkers listed in Table 3.

INSM1 is an intronless gene which encodes a protein containing both a zinc finger DNA-binding domain and a putative prohormone domain whose function is largely unknown. INSM1 protein interacts with SORBS1, a CAP/Ponsin protein that regulates cell adhesion, growth factor signaling and cytoskeletal formation in skeletal muscle, liver, and adipose tissue.

GADD45G is a member of a group of genes whose transcript levels are increased following exposure to a stress arrest condition and treatment with DNA damaging agents. The protein encoded by this gene responds to environmental stresses by mediating activation of the p38/JNK pathway via MTK1/MEKK4 kinase. GADD45G is, in turn, regulated by NF-κB. GADD45G is down-regulated in various types of cancerous cell due to methylation of the GADD45G promotor. The low expression can also be explained by increased NF-κB activation.

C1QL1 is complement component 1, q subcomponent-like 1 and its physiological function is largely unknown. It is a member of the C1Q domain proteins which have important signaling roles in inflammation and in adaptive immunity.

TABLE 1
High Expression Biomarkers

| Gene | Accession | Description |
|---|---|---|
| INSM1 | NM_002196 | Insulinoma-associated protein 1 |
| GADD45G | NM_006705 | Growth arrest and DNA-damage- |
| | | inducible protein GADD45 gamma |
| C1QL1 | NM_006688 | Complement component 1, q |
| | | subcomponent-like 1 |

*Genes in Table 1 are highly expressed in the SCLC subtype sensitive to pentamidine as compared to normal or other SCLC subtypes. Expression of these genes are down-regulated upon treatment with pentamidine.

In the present invention, the biomarker listed in Table 2 is either not expressed at a detectable level or exhibits a similar expression level to the ones observed in a normal control lung tissue. In one embodiment, the biomarker listed in Table 2 is expressed at a low level as compared to a control.

STAT6 encodes a protein that is a member of the STAT family of transcription factors. In response to cytokines and growth factors, STAT family members are phosphorylated by the receptor associated kinases, and then form homo- or heterodimers that translocate to the cell nucleus where they act as transcription activators. This protein plays a central role in exerting IL4 mediated biological responses. It is found to induce the expression of BCL2L1/BCL-X(L), which is responsible for the anti-apoptotic activity of IL4. Knockout studies in mice suggested the roles of this gene in differentiation of T helper 2 (Th2) cells, expression of cell surface markers, and class switch of immunoglobulins.

SMAD3 is a member of Smad family, an essential intracellular signaling component of the transforming growth factor-β (TGF-β) superfamily involved in a range of biological activities. SMAD3 along with SMAD2 have been identified as receptor-activated SMADs for TGF-β signaling and has been the focus of intensive studies. Although no definite differences in regulation or function have been established between these TGF-β signaling molecules, the expression of SMAD3, but not its close relative, SMAD2, is down-regulated by TGF-β mediated signals themselves in human lung epithelial cells. This down-regulation of Smad3 by TGF-β does not result from shortening of the half-life of SMAD3 mRNA. Constitutive expression of Smad3 in the presence of TGF-β induced apoptotic cell death, with an adverse effect on the cell growth of human lung epithelial cells. Apoptotic cell death could also be induced by forced expression of Smad2 in the presence of TGF-β, but less efficiently than by that of Smad3.

RNF43 encodes a RING-type E3 ubiquitin ligase and is predicted to contain a transmembrane domain, a protease-associated domain, an ectodomain, and a cytoplasmic RING domain. This protein is thought to negatively regulate Wnt signaling, and expression of this gene results in an increase in ubiquitination of Frizzled receptors, an alteration in their subcellular distribution, resulting in reduced surface levels of these receptors. Mutations in this gene have been reported in multiple tumor cells, including colorectal and endometrial cancers. Alternative splicing results in multiple transcript variants encoding different isoforms.

TABLE 2
Low Expression Biomarkers

| Gene | Accession | Description |
|---|---|---|
| STAT6 | NM_001178078 | Signal transducer and |
| | NM_001178079 | activator of transcription 6 |
| | NM_001178080 | |
| | NM_001178081 | |
| | NM_003153 | |
| SMAD3 | NM_001145102 | Mothers against |
| | NM_001145103 | decapentaplegic homolog 3 |
| | NM_001145104 | (SMAD family member 3) |
| | NM_005902 | |
| RNF43 | NM_017763 | RING-type E3 ubiquitin |
| | | ligase |

*Genes in Table 2 exhibit no expression or are expressed at low levels relative to a normal control or other SCLC subtype, but are activated upon treatment with pentamidine.

TABLE 3
All genes whose expression are significantly elevated in a subtype
of small cell lung cancer predicted to be sensitive to pentamidine.

| Gene | RNA Accession No. | Description |
|---|---|---|
| APLP1 | NM_001024807 | Amyloid beta precursor like protein 1 |
| SEZ6L2 | NM_001114099 | Seizure related 6 homolog like 2 |
| | NM_001107571 | |
| LHX2 | NM_004789.3 | LIM homeobox2 |
| | NM_004780.3 | |
| SMAD9 | NM_001127217.2 | SMAD family member 9 |
| DPF1 | NM_001135155.2 | double PHD fingers 1 |
| NBEA | NM_001204197.1 | Neurobeachin |
| MPPED1 | NM_001044370.1 | Metallophosphoesterase |
| | | domain containing 1 |
| PCSK1N | NM_013271.4 | Proprotein convertase subtilisin/ |
| | | kexin type 1 inhibitor |
| WSCD1 | XM_005256573.1 | WSC domain containing 1 |
| RALYL | NM_001100391.2 | RALY RNA binding protein like |
| H2AFX | NM_002105.2 | H2A histone family member X |
| TUBB3 | NM_001197181.1 | Tubulin beta 3 class III |
| GABRD | NM_000815.4 | Gamma-aminobutyric acid type |
| | | A receptor delta subunit |
| HOXB8 | XM_017024564 | Homeobox B8 |
| DLX2 | NM_004405.3 | Distal-less homeobox2 |
| CLDN11 | NM_001185056.1 | Claudin 11 |
| CKB | NM_001823.4 | Creatine kinase B |
| ID4 | NM_001546.3 | Inhibitor of DNA binding 4, HLH protein |
| SBNO2 | XM_011527804.2 | Strawberry notch homolog 2 |
| FZD9 | NM_003508.2 | Frizzled class receptor 9 |
| AKR1C3 | NM_001253908 | Aldo-keto reductase family 1 member C3 |
| | NM_001253909 | |
| | NM_003739.5 | |
| MTAP | NM_002451.3 | Methylthioadenosine phosphorylase |
| SNCAIP | XM_006714734.2 | Synuclein alpha interacting protein |
| | XM_011543738.2 | |
| | XM_017010083.1 | |
| | XM_011543739.1 | |
| | XM_005272139.1 | |
| | XM_017010085.1 | |
| DLX5 | NM_005221.5 | Distal-less homeobox 5 |
| CADPS | NM_003716.3 | Calcium dependent secretion activator |
| | NM_183393.2 | |
| | NM_183384.2 | |
| DGKB | NM_001350705.1 | Diacylglycerol kinase beta |
| | NM_001350706.1 | |
| | NM_001350707.1 | |
| | NM_001350708.1 | |
| | NM_001350709.1 | |
| | NM_001350711.1 | |
| | NM_001350712.1 | |
| | NM_001350713.1 | |
| | NM_001350714.1 | |
| LFNG | NM_001040167.1 | LFNG O-fucosylpeptide 3-beta-N- |
| | NM_001040168.1 | acetylglucosaminyltransferase |
| | NM_001166355.1 | |
| | NM_002304.2 | |
| | NM_002304.2 | |
| CER1 | NM_005454.2 | Cerberus 1, DAN family BMP antagonist |
| KIAA0319 | NM_001168374 | KIAA0319 |
| VWA5B2 | NM_001320373.1 | von Willebrand factor A |
| | NM_138345.2 | domain containing 5B2 |
| SLC22A17 | NM_001289050 | Solute carrier family 22 member 17 |
| | NM_016609.4 | |
| | NM_020372.3 | |
| FOXA2 | NM_021784.4 | Forkhead box A2 |
| | NM_153675.2 | |
| HNRNPA0 | NM_006805.3 | Heterogeneous nuclear |
| | | ribonucleoprotein A0 |
| HELLS | NM_001289067.1 | Helicase, lymphoid specific |
| | NM_001289068.1 | |
| | NM_001289069.1 | |
| | NM_001289070.1 | |
| | NM_001289071.1 | |
| | NM_001289072.1 | |
| | NM_001289073.1 | |
| | NM_001289074.1 | |
| | NM_001289075.1 | |
| | NM_018063.4 | |
| NRTN | XM_011528041.2 | Neurturin |
| SOX21 | NM_007084.3 | SRY-Box21 |

*Genes in Table 3 are highly expressed in SCLC subtype responsive to pentamidine, as compared to normal or other SCLC subtypes. Expression of these genes are down-regulated upon treatment with pentamidine.

In some embodiments, the predictive gene expression signature may include at least two biomarkers comprising INSM1 and one or more biomarkers selected from the group consisting of GADD45G, STAT6, SMAD3, C1QL1, and RNF43 and optionally at least one biomarker selected from the group of biomarkers listed in Table 3.

TABLE 4
Precision and Recall Rates for Different Combinations of Biomarkers

| Biomarkers | Prcn | Recall | Algorithm |
|---|---|---|---|
| INSM1, GADD45G, RNF43 | 0.961 | 0.983 | Logistic regression |
| INSM1, C1QL1, RNF43 | 0.961 | 0.930 | Logistic regression |
| INSM1, GADD45G, C1QL1, RNF43 | 0.961 | 0.979 | Logistic regression |
| INSM1, GADD45G, STAT6, RNF43 | 0.961 | 0.896 | Logistic regression |
| INSM1, C1QL1, STAT6, RNF43 | 0.961 | 0.938 | Logistic regression |
| INSM1, GADD45G, C1QL1, STAT6, RNF43 | 0.961 | 0.892 | Logistic regression |
| INSM1, GADD45G, C1QL1, SMAD3, RNF43 | 0.961 | 0.959 | Logistic regression |
| INSM1, GADD45G, C1QL1, STAT6, SMAD3, RNF43 | 0.961 | 0.915 | Logistic regression |
| INSM1, GADD45G, C1QL1, STAT6, SMAD3, RNF43 | 0.958 | 0.833 | Naïve Bayes |
| INSM1, C1QL1, STAT6, SMAD3, RNF43 | 0.957 | 0.850 | Naïve Bayes |
| INSM1, STAT6, RNF43 | 0.956 | 0.950 | Logistic regression |
| INSM1, SMAD3, RNF43 | 0.951 | 0.829 | Naïve Bayes |
| INSM1, GADD45G, STAT6, RNF43 | 0.951 | 0.716 | Naïve Bayes |
| INSM1, STAT6, RNF43 | 0.948 | 0.809 | Naïve Bayes |
| INSM1, SMAD3, RNF43 | 0.946 | 0.901 | Logistic regression |
| INSM1, GADD45G, SMAD3, RNF43 | 0.946 | 0.963 | Logistic regression |
| INSM1, C1QL1, SMAD3, RNF43 | 0.946 | 0.905 | Logistic regression |
| INSM1, STAT6, SMAD3, RNF43 | 0.946 | 0.872 | Logistic regression |
| INSM1, GADD45G, STAT6, SMAD3, RNF43 | 0.946 | 0.871 | Logistic regression |
| INSM1, C1QL1, STAT6, SMAD3, RNF43 | 0.946 | 0.856 | Logistic regression |
| INSM1, C1QL1, SMAD3, RNF43 | 0.937 | 0.923 | Naïve Bayes |
| INSM1, STAT6 | 0.899 | 0.922 | Logistic regression |
| INSM1, SMAD3 | 0.899 | 0.885 | Logistic regression |
| INSM1, C1QL1, STAT6 | 0.899 | 0.918 | Logistic regression |
| INSM1, C1QL1, SMAD3 | 0.899 | 0.864 | Logistic regression |
| INSM1, STAT6, SMAD3 | 0.899 | 0.868 | Logistic regression |
| INSM1, C1QL1, STAT6, SMAD3 | 0.899 | 0.856 | Logistic regression |
| INSM1, GADD45G, C1QL1, SMAD3 | 0.895 | 0.834 | Logistic regression |
| INSM1, GADD45G, C1QL1, STAT6, SMAD3 | 0.895 | 0.814 | Logistic regression |
| INSM1, SMAD3 | 0.894 | 0.963 | Naïve Bayes |
| INSM1, GADD45G, STAT6, SMAD3 | 0.891 | 0.870 | Naïve Bayes |
| INSM1, GADD45G, C1QL1, RNF43 | 0.883 | 0.854 | Decision tree |
| INSM1, GADD45G, STAT6, RNF43 | 0.883 | 0.868 | Decision tree |

* Variation ranging from 0.6 to 1.0 for precision and recall.

Expression levels of the genes listed in Table 1 are found to be high in tumors of the subtype 1, and can be used as a distinguishing feature of the SCLC subtype sensitive to pentamidine or a pharmaceutically acceptable salt of pentamidine from the rest of the small cell lung cancers. Upon treatment with pentamidine, however, expression patterns of these genes are repressed nearly to normal levels. On the other hand, the genes listed in Table 2 exhibit low levels of expression in tumors of a SCLC subtype before treatment with pentamidine. Upon treatment with pentamidine, however, expression of these genes indeed increased nearly to normal levels, rendering them pentamidine responsive genes. Accordingly, expression profiles of these biomarkers not only allows for identification of the pentamidine sensitive/responsive subtype from the rest of SLCL subtypes, but also function as a marker or indicator for disease progression during the treatment with pentamidine or a pharmaceutically acceptable salt thereof (e.g., pentamidine isethionate, pentamidine gluconate, or pentamidine mesylate) in a subject having small cell lung cancer.

As described herein, expression can be readily determined directly or indirectly by any suitable method. In some embodiments, determining the level of expression comprises assaying a nucleic acid or protein level in a small cell lung cancer sample from said subject. In some embodiments, determining the level of expression comprises assaying a nucleic acid in a small cell lung cancer sample from said subject. In some embodiments, the nucleic acid is RNA. In some embodiments, determining the level of expression comprises assaying a protein level in a small cell lung cancer sample from said subject. In certain embodiments, the level of expression of the biomarker is determined at the nucleic acid level using any suitable method. For example, the level of expression of the biomarker can be determined by detecting cDNA, mRNA or DNA. In particular embodiments, the level of expression of the biomarker is determined by using a technique selected from the group consisting of polymerase chain reaction (PCR) amplification reaction, reverse-transcriptase PCR analysis, quantitative reverse transcriptase PCR analysis, microarray, RNA-seq, Northern blot analysis, RNAase protection assay, digital RNA detection/quantitation (e.g., nanoString) and combinations or sub-combinations thereof.

In some embodiments, the biomarker is expressed at a level at least 2 times greater than expression level(s) measured in one or more controls, such as at least 2, 3, 4, 5, 6, 7, 8, 9, 10, 15, 20, 25, 30, 35, 40, or 45 times greater. In some embodiments, the biomarker is expressed at a level at least 3 times greater than expression level(s) measured in one or more controls. In some embodiments, the biomarker is expressed at a level at least 4 times greater than expression level(s) measured in one or more controls. In some embodiments, the biomarker is expressed at a level at least 5 times greater than expression level(s) measured in one or more controls. In some embodiments, the biomarker is expressed at a level at least 10 times greater than expression level(s) measured in one or more controls. In some embodiments, the biomarker is expressed at a level 2-50 times greater than expression level(s) measured in one or more controls, such as 3-45, 4-45, 5-45, 6-45, 7-45, 8-45, 9-45, 10-45, 15-45, 20-45, 25-45, 30-45, 35-45, or 40-45 times greater.

In some embodiments, the biomarker is a protein. In some embodiments, the protein is expressed at a level at least 2 times greater than expression level(s) measured in one or more controls, such as at least 2, 3, 4, 5, 6, 7, 8, 9, 10, 15, 20, 25, 30, 35, 40, or 45 times greater. In some embodiments, the protein is expressed at a level at least 3 times greater than expression level(s) measured in one or more controls. In some embodiments, the protein is expressed at a level at least 4 times greater than expression level(s) measured in one or more controls. In some embodiments, the protein is expressed at a level at least 5 times greater than expression level(s) measured in one or more controls. In some embodiments, the protein is expressed at a level at least 10 times greater than expression level(s) measured in one or more controls. In some embodiments, the protein is expressed at a level 2-50 times greater than expression level(s) measured in one or more controls, such as 3-45, 4-45, 5-45, 6-45, 7-45, 8-45, 9-45, 10-45, 15-45, 20-45, 25-45, 30-45, 35-45, or 40-45 times greater.

In some embodiments, the biomarker is a nucleic acid. In some embodiments, the nucleic acid is expressed at a level at least 2 times greater than expression level(s) measured in one or more controls, such as at least 2, 3, 4, 5, 6, 7, 8, 9, 10, 15, 20, 25, 30, 35, 40, or 45 times greater. In some embodiments, the nucleic acid is expressed at a level at least 3 times greater than expression level(s) measured in one or more controls. In some embodiments, the nucleic acid is expressed at a level at least 4 times greater than expression level(s) measured in one or more controls. In some embodiments, the nucleic acid is expressed at a level at least 5 times greater than expression level(s) measured in one or more controls. In some embodiments, the nucleic acid is expressed at a level at least 10 times greater than expression level(s) measured in one or more controls. In some embodiments, the nucleic acid is expressed at a level 2-50 times greater than expression level(s) measured in one or more controls, such as 3-45, 4-45, 5-45, 6-45, 7-45, 8-45, 9-45, 10-45, 15-45, 20-45, 25-45, 30-45, 35-45, or 40-45 times greater.

In some embodiments, the biomarker is expressed at a level that is less than 100% of expression level(s) measured in one or more controls, such as less than 95%, 90%, 80%, 70%, 60%, 50%, 40%, 30%, 20%, 10%, 5%, or 1%. In some embodiments, the biomarker is expressed at a level less than 95% of the expression level(s) measured in one or more controls. In some embodiments, the biomarker is expressed at a level less than 90% of expression level(s) measured in one or more controls. In some embodiments, the biomarker is expressed at a level less than 70% of expression level(s) measured in one or more controls. In some embodiments, the biomarker is expressed at a level less than 50% of expression level(s) measured in one or more controls. In some embodiments, the biomarker is expressed at a level that is 1%-100% of the expression level(s) measured in one or more controls, such as 5%-95%, 10%-90%, 20%-80%, or 30%-70% of the expression level(s) measured in one or more controls.

### Subjects

The methods disclosed herein are generally used to reduce, treat or eliminate small cell lung cancer in a subject. The subject may be any human patient, particularly a cancer patient suffering from small cell lung cancer. In some cases, the patient is in a particular stage of small cell lung cancer treatment. Examples of cancer include cancers that cause solid tumors. Furthermore, any of the cancers mentioned herein may be a primary cancer (e.g., a cancer that is named after the part of the body where it first started to grow) or a secondary, metastatic cancer (e.g., cancer that has originated from another part of the body). In one embodiment, the subject is a human patient suffering from metastatic small cell lung cancer. In another embodiment, the subject is a human patient suffering from stage I small cell lung cancer. In another embodiment, the subject is a human patient suffering from stage II small cell lung cancer. In another embodiment, the subject is a human patient suffering from stage III small cell lung cancer.

### Drug Administration

Pentamidine or a pharmaceutically acceptable salt thereof (e.g., pentamidine isethionate, pentamidine gluconate, or pentamidine mesylate) can be administered to a subject suffering from a subtype of small cell lung cancer having a predicted gene expression signature intravenously (e.g., by injection or infusion), intramuscularly, subcutaneously, intrathecally, orally, or by inhalation. In one embodiment, pentamidine or a pharmaceutically acceptable salt of pentamidine (e.g., pentamidine isethionate, pentamidine gluconate, or pentamidine mesylate) is administered to a human patient in an effective amount intravenously. In another embodiment, pentamidine or a pharmaceutically acceptable salt of pentamidine (e.g., pentamidine isethionate, pentamidine gluconate, or pentamidine mesylate) is administered to a human patient in an effective amount orally. In one embodiment, pentamidine or a pharmaceutically acceptable salt of pentamidine (e.g., pentamidine isethionate, pentamidine gluconate, or pentamidine mesylate) is administered to a human patient in an effective amount by inhalation.

The treatment regimen for pentamidine or a pharmaceutically acceptable salt thereof (e.g., pentamidine isethionate, pentamidine gluconate, or pentamidine mesylate) typically includes at least one of the following parameters and more typically includes many or all of the following parameters: the formulation, the dosage, the route of administration and/or the frequency of administration. Selection of the particular parameters of the treatment regimen can be based on known treatment parameters for pentamidine previously established in the art such as those described in the Dosage and Administration protocols set forth in the FDA Approved Label for NeubuPen™, Pentam 300™ and Pentacarinat™, the entire contents of which are incorporated herein by reference. For example, pentamidine isethionate can be administered intravenously or intramuscularly on every day for a 14-21 day cycle, for example at a dose of 4.0 mg per kg of patients or if a dose reduction is indicated (e.g., for hepatic or renal impairment), at a dose of 3.0 mg per kg or 2.0 mg per kg. Various modifications to dosage, formulation, route of administration and/or frequency of administration can be made based on various factors including, for example, the disease, age, sex, and weight of the patient, as well as the severity or stage of cancer (see, for example, U.S. Pat. Nos. 6,653,341 and 6,469,182, the entire contents of each of which are hereby incorporated herein by reference).

As used herein, the term “therapeutically effective amount” means an amount of pentamidine, or a pharmaceutically acceptable salt thereof (e.g., pentamidine isethionate, pentamidine gluconate, or pentamidine mesylate) as described herein, that is capable of treating small cell lung cancer. The dose of a compound to be administered according to this invention will be determined in light of the particular circumstances surrounding the case including, for example, the compound administered, the route of administration, condition of the patient, and the pathological condition being treated, for example, the stage of small cell lung cancer. In one embodiment, pentamidine or a pharmaceutically acceptable salt thereof (e.g., pentamidine isethionate, pentamidine gluconate, or pentamidine mesylate) can be administered intravenously at a dose of, for example, 0.5, 0.6, 0.7, 0.8, 0.9, 1.0, 1.5, 2.0, 3.0, 4.0, 5.0, 6.0, 7.0, 8.0, 9.0, 10, 11, 12, 13, 14, 15, 16, 17, 18, 19, 20, 21, 22, 23, 24, 25, 26, 27, 28, 29, or 30 mg per kg per injection. In one embodiment, pentamidine or a pharmaceutically acceptable salt thereof (e.g., pentamidine isethionate, pentamidine gluconate, or pentamidine mesylate) can be administered intravenously at a daily dose of, for example, about 0.5 mg per kg, about 1.0 mg per kg, about 1.5 mg per kg, about 2.0 mg per kg, about 5 mg per kg, about 6 mg per kg, about 7 mg per kg, about 8 mg per kg, about 9 mg per kg, about 10 mg per kg, about 11 mg per kg, about 12 mg per kg, about 13 mg per kg, about 14 mg per kg, about 15 mg per kg, about 16 mg per kg, about 17 mg per kg, about 18 mg per kg, about 19 mg per kg, about 20 mg per kg, about 21 mg per kg, about 22 mg per kg, about 23 mg per kg, about 24 mg per kg, about 25 mg per kg, about 26 mg per kg, about 27 mg per kg, about 28 mg per kg, about 29 mg per kg, or about 30 mg per kg of patient.

For administration to a subject, pentamidine, or a pharmaceutically acceptable salt thereof (e.g., pentamidine isethionate, pentamidine gluconate, or pentamidine mesylate), typically is formulated into a pharmaceutical composition comprising pentamidine, or a pharmaceutically acceptable salt thereof (e.g., pentamidine isethionate, pentamidine gluconate, or pentamidine mesylate), and a pharmaceutically acceptable carrier. Therapeutic compositions typically should be sterile and adequately stable under the conditions of manufacture and storage.

There are numerous types of anti-cancer approaches that can be used in conjunction with pentamidine, or a pharmaceutically acceptable salt thereof (e.g., pentamidine isethionate, pentamidine gluconate, or pentamidine mesylate) treatment, according to the invention. These include, for example, treatment with chemotherapeutic agents, biological agents, radiation, and surgery. The methods of the invention can employ these approaches to treat the same types of cancers as those for which they are known in the art to be used, as well as others, as can be determined by those of skill in this art. Also, these approaches can be carried out according to parameters (e.g., regimens and doses) that are similar to those that are known in the art for their use. However, as is understood in the art, it may be desirable to adjust some of these parameters, due to the additional use of pentamidine, or a pharmaceutically acceptable salt thereof (e.g., pentamidine isethionate, pentamidine gluconate, or pentamidine mesylate), with these approaches. For example, if a drug such as etoposide or cisplatin is normally administered as a sole therapeutic agent, when combined with pentamidine, according to the invention, it may be desirable to decrease the dosage of the drug, as can be determined by those of skill in this art.

Chemotherapeutic drugs of several different types including, for example, antimetabolites, antibiotics, alkylating agents, plant alkaloids, hormonal agents, anticoagulants, antithrombotics, and other natural products, among others, can be used in conjunction with pentamidine, or a pharmaceutically acceptable salt thereof, according to the invention. A pharmaceutical composition comprising pentamidine or a pharmaceutically acceptable salt thereof (e.g., pentamidine isethionate, pentamidine gluconate, or pentamidine mesylate) can be administered in a combination therapy, i.e., combined with other agents, such as etoposide, cisplatin, oxaliplatin, gemcitabine, irinotecan, or taxol (see, for example, U.S. Patent Application Publication No. 20120128667, the entire contents of which are hereby incorporated herein by reference).

Numerous approaches for administering anti-cancer drugs are known in the art, and can readily be adapted for use in the present invention. In the case that one or more drugs are to be administered in conjunction with pentamidine or a pharmaceutically acceptable salt thereof (e.g., pentamidine isethionate, pentamidine gluconate, or pentamidine mesylate), for example, the drugs can be administered together, in a single composition, or separately, as part of a comprehensive treatment regimen. For systemic administration, the drugs can be administered by, for example, intravenous injection or infusion (continuous or bolus). Appropriate scheduling and dosing of such administration can readily be determined by those of skill in this art based on, for example, preclinical studies in animals and clinical studies (e.g., phase I studies) in humans. Many regimens used to administer chemotherapeutic drugs involve, for example, intravenous administration of a drug (or drugs) followed by repetition of this treatment after a period (e.g., 1-4 weeks) during which the patient recovers from any adverse side effects of the treatment. It may be desirable to use both drugs at each administration or, alternatively, to have some (or all) of the treatments include only one drug.

The compounds of the current disclosure may be administered by any of the accepted modes of administration of agents having similar utilities, for example, by cutaneous, oral, topical, intradermal, intrathecal, intravenous, subcutaneous, intramuscular, intra-articular, intraspinal or spinal, nasal, epidural, or transdermal/transmucosal inhalable routes. The most suitable route will depend on the nature and severity of the condition being treated.

In a particular example, the pharmaceutical composition provided herein may be administered to a patient orally. In another particular example, the pharmaceutical composition comprising pentamidine or a pharmaceutically acceptable salt thereof may be administered to a patient intravenously (via, e.g., injection or infusion). In another particular example, the pharmaceutical composition comprising pentamidine or a pharmaceutically acceptable salt thereof may be administered to a patient intramuscularly. In a particular example, the pharmaceutical composition comprising pentamidine or a pharmaceutically acceptable salt thereof may be administered to a patient nasally.

A pharmaceutical composition (e.g., for oral administration or for inhalation, injection, infusion, subcutaneous delivery, intramuscular delivery, intraperitoneal delivery, sublingual delivery, or other methods) may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. A liquid pharmaceutical composition may be delivered orally.

A pharmaceutical composition comprising pentamidine or a pharmaceutically acceptable salt thereof (e.g., pentamidine isethionate, pentamidine gluconate, or pentamidine mesylate) may be formulated for sustained or slow release (also called timed release or controlled release). Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. Non-limiting examples of excipients include water, alcohol, glycerol, chitosan, alginate, chondroitin, Vitamin E, mineral oil, and dimethyl sulfoxide (DMSO). The amount of compound contained within a sustained release formulation depends upon the site of implantation, the rate and expected duration of release, and the nature of the condition, disease or disorder to be treated or prevented.

The pharmaceutical composition comprising pentamidine or a pharmaceutically acceptable salt thereof (e.g., pentamidine isethionate, pentamidine gluconate, or pentamidine mesylate) may be effective over time. In some cases, the pharmaceutical composition may be effective for one or more days. In some cases, the duration of efficacy of the pharmaceutical composition is over a long period of time. In some cases, the efficacy of the pharmaceutical composition may be greater than 2 days, 3 days, 4 days, 5 days, 6 days, 1 week, 2 weeks, 3 weeks, or 1 month.

In making the pharmaceutical composition comprising pentamidine or a pharmaceutically acceptable salt thereof (e.g., pentamidine isethionate, pentamidine gluconate, or pentamidine mesylate), the active ingredient can be diluted by an excipient. Some examples of suitable excipients include lactose, dextrose, sucrose, sorbitol, mannitol, starches, gum acacia, calcium phosphate, alginates, tragacanth, gelatin, calcium silicate, microcrystalline cellulose, PEG, polyvinylpyrrolidone, cellulose, water, sterile saline, syrup, and methyl cellulose. The compositions of the disclosure can be formulated so as to provide quick, sustained or delayed release of the active ingredient after administration to the patient by employing procedures known in the art. In some cases, the pharmaceutical composition comprising pentamidine or a pharmaceutically acceptable salt thereof (e.g., pentamidine isethionate, pentamidine gluconate, or pentamidine mesylate) may comprise an excipient that can provide long term preservation, bulk up a formulation that contains potent active ingredients, facilitate drug absorption, reduce viscosity, add flavoring, or enhance the solubility of the pharmaceutical composition.

In some cases, the pharmaceutical composition comprising pentamidine or a pharmaceutically acceptable salt thereof (e.g., pentamidine isethionate, pentamidine gluconate, or pentamidine mesylate) may comprise a pharmaceutically acceptable carrier. The pharmaceutically acceptable carrier may include any and all solvents, dispersion media, coatings, antibacterial and antifungal agents, isotonic and absorption delaying agents, and the like that are physiologically compatible. Preferably, the carrier is suitable for parenteral (e.g., intravenous, intramuscular, subcutaneous, intrathecal) administration (e. g., by injection or infusion). Depending on the route of administration, the active compound may be coated in a material to protect the compound from the action of acids and other natural conditions that may inactivate the compound.

Also provided herein are pharmaceutical compositions for use in any of the methods described herein. Also provided herein are uses of comprising pentamidine or a pharmaceutically acceptable salt thereof for the manufacture of a medicament for the treatment of a disease disclosed herein according to a method disclosed herein.

### Kits

The invention also provides kits comprising one or more materials for predicting whether pentamidine or a pharmaceutically acceptable salt thereof (e.g., pentamidine isethionate, pentamidine gluconate, or pentamidine mesylate) may be used to treat a subject having small cell lung cancer. These kits include reagents for determining the level of expression of at least one, for example, 1, 2, 3, 4, 5, 6, 7, 8, 9 or 10, biomarker(s) selected from the group biomarkers listed in Table 1, Table 2 and/or Table 3, instructions for use of the kit to predict whether pentamidine or a pharmaceutically acceptable salt thereof may be used to treat a subject having small cell lung cancer.

The kits may optionally comprise additional components useful for performing the methods of the invention. By way of example, the kits may comprise reagents for obtaining a biological sample from a subject, a control sample, and/or pentamidine or a pharmaceutically acceptable salt thereof.

In some cases, the kit may comprise biomarker testing materials, wherein the testing materials may exist as a distinct component within the kit. For example, the testing materials and reagents for determining expression levels of the biomarker can be a probe for identifying expression of the biomarker in the samples. The reagents for determining the level of expression of the biomarker can be a probe for amplifying and/or detecting the biomarker. In yet another embodiment, the reagent for determining the level of expression of the biomarker can be an antibody, for example, an antibody specific for the product of the expression of the biomarkers described herein.

In one embodiment, the reagents for determining the expression level of at least one biomarker in a biological sample from the subject comprises a nucleic acid preparation sufficient to detect expression of a nucleic acid, for example, mRNA, encoding the biomarker. This nucleic acid preparation includes at least one, and may include more than one, nucleic acid preparation can detect the expression of nucleic acid, for example, mRNA, encoding the biomarker in the sample from the subject. A preferred nucleic acid preparation includes two or more PCR primers that allow for PCR amplification of a segment of the mRNA encoding the biomarker of interest. In other embodiments, the kit includes a nucleic acid preparation for each of at least 1, 2, 3, 4, 5, 6, 7, 8, 9 or 10 biomarkers provided in Table 1, Table 2 and/or Table 3.

Alternatively, the reagents for detecting expression levels in the subject of one or more biomarkers predictive of susceptibility to treatment with pentamidine or a pharmaceutically acceptable salt thereof (e.g., pentamidine isethionate, pentamidine gluconate, or pentamidine mesylate) can comprise a reagent that detects the gene product of the nucleic acid encoding the biomarker(s) of interest sufficient to distinguish it from other gene products in a sample from the subject. A non-limiting example of such a reagent is a monoclonal antibody preparation (comprising one or more monoclonal antibodies) sufficient to detect protein expression of at least one biomarker protein encoded by the genes listed in Table 1, Table 2 and/or Table 3 in a sample from the subject, such as a peripheral blood mononuclear cell sample.

The means for determining the expression level of the biomarkers of Table 1, Table 2, and/or Table 3 can also include, for example, buffers or other reagents for use in an assay for evaluating expression (e.g., at either the nucleic acid or protein level).

In one embodiment, the kit may further comprise pentamidine or a pharmaceutically acceptable salt thereof (e.g., pentamidine isethionate, pentamidine gluconate, or pentamidine mesylate) for treating small cell lung cancer, as described herein. In some cases, the kit may comprise pentamidine, or a pharmaceutically acceptable salt thereof (e.g., pentamidine isethionate, pentamidine gluconate, or pentamidine mesylate), and testing materials for one or more biomarkers described herein. In some embodiments, the kits with unit doses of pentamidine described herein, usually in oral, injectable or inhalable doses, are provided. Such kits may include a container containing the unit dose, an informational package insert describing the use and attendant benefits of pentamidine, or a pharmaceutically acceptable salt thereof (e.g., pentamidine isethionate, pentamidine gluconate, or pentamidine mesylate) in treating small cell lung cancer, and optionally a device for delivery of pentamidine, or a pharmaceutically acceptable salt thereof (e.g., pentamidine isethionate, pentamidine gluconate, or pentamidine mesylate).

In one embodiment, the kit may comprise reagents for obtaining a biological sample from a subject. The kit may comprise vials, tubes, needles, an inhaling device comprising pentamidine or a pharmaceutically acceptable salt thereof, and/or other biomarker testing materials described herein.

In some cases, the kit may include a control sample. In one embodiment, the kit may include a positive control sample and/or a negative control sample. In one embodiment, the kit may include a positive control sample that exhibits a predicted gene expression signature for pentamidine susceptibility. In another embodiment, the kit includes a negative control sample that does not exhibit the predicted gene expression signature for pentamidine susceptibility. In one embodiment, the kit includes a positive and negative control samples.

The kit may further comprise any device suitable for administration of the pharmaceutical composition comprising pentamidine or a pharmaceutically acceptable salt thereof. For example, a kit comprising an injectable formulation of pharmaceutical compositions may comprise a needle suitable for intravenous or subcutaneous administration and an alcohol wipe for sterilization of the injection site.

In some cases, kits may be provided with instructions. The instructions may provide information on how to use the compositions of the present disclosure. The instructions may further provide information on how to use the devices of the present disclosure. The instructions may provide information on how to perform the methods of the disclosure. In some cases, the instructions may provide dosing information. The instructions may provide drug information such as the mechanism of action, the formulation of the drug, adverse risks, contraindications, and the like. In some cases, the kit is purchased by a physician or health care provider for administration at a clinic or hospital. In some cases, the kit is purchased by a laboratory and used for screening candidate compounds. Preferably, the kit is designed for use with a human patient.

In another aspect, the present invention provides methods of treating a subtype of small cell lung cancer using a kit for predicting whether pentamidine, or a pharmaceutically acceptable salt thereof (e.g., pentamidine isethionate, pentamidine gluconate, or pentamidine mesylate), may be used to treat a subject having small cell lung cancer, including testing materials and reagents for determining the level of expression of a biomarker selected from the group of biomarkers listed in Table 1, Table 2 or Table 3; and instructions for use of the kit to predict whether pentamidine, or a pharmaceutically acceptable salt thereof (e.g., pentamidine isethionate, pentamidine gluconate, or pentamidine mesylate), may be used to treat a subject having small cell lung cancer.

### Exemplary Embodiments

Among the provided embodiments are:

Embodiment 1. A method of treating a subject suffering from small cell lung cancer, the method comprising
a. assaying a small cell lung cancer sample derived from said subject to determine the level of expression in said sample of at least one biomarker selected from the group of biomarker genes listed in Table 1 and optionally at least one biomarker from Table 2;
b. detecting a high level of expression of said at least one biomarker in Table 1 and optionally a low level of expression of said at least one biomarker in Table 2 in said sample relative to a normal control; and
c. administering a therapeutically effective amount of pentamidine or a pharmaceutically acceptable salt thereof, to said subject.

Embodiment 2. The method of embodiment 1, wherein the pharmaceutically acceptable salt of pentamidine is pentamidine isethionate.

Embodiment 3. The method of embodiment 1, wherein the pharmaceutically acceptable salt of pentamidine is pentamidine mesylate.

Embodiment 4. The method of embodiment 1, wherein the pharmaceutically acceptable salt of pentamidine is pentamidine gluconate.

Embodiment 5. The method of any one of embodiments 1-4, wherein said at least one biomarker from Table 1 is INSM1.

Embodiment 6. The method of any one of embodiments 1-5, wherein said small cell lung cancer is Stage I, II or III small cell lung cancer.

Embodiment 7. The method of any one of embodiments 1-6, wherein at least 2 or 3 biomarkers selected from the group of biomarkers listed in Table 1 have a high level of expression as compared to a control.

Embodiment 8. The method of any one of embodiments 1-7, wherein said at least one biomarker selected from the group of biomarkers listed in Table 2 has a low level of expression as compared to a control.

Embodiment 9. The method of embodiment 8, wherein said a least one biomarker selected from the group of biomarkers listed in Table 2 is not expressed at a detectable level.

Embodiment 10. The method of any one of embodiments 1-9, wherein the level of expression of said biomarker is determined by detecting mRNA levels.

Embodiment 11. The method of any one of embodiments 1-9, wherein the level of expression of said biomarkers is determined at the protein level.

Embodiment 12. The method of embodiment 11, wherein the presence of the protein is detected using an antibody that binds to the protein.

Embodiment 13. The method of embodiment 12, wherein the antibody is labeled.

Embodiment 14. The method of embodiment 11 or 12, wherein the level of expression of said biomarker is determined by immunoassay, a western blot assay, immunofluorimetry, ELISA assay, electrochemiluminescence assay, or immunoprecipitation.

Embodiment 15. The method of any one of embodiments 1-14, wherein said subject is a human patient.

Embodiment 16. The method of any one of embodiments 1-15, wherein said pentamidine or pharmaceutically acceptable salt thereof is administered to the subject orally.

Embodiment 17. The method of any one of embodiments 1-15, wherein said pentamidine or pharmaceutically acceptable salt thereof is administered to the subject by inhalation.

Embodiment 18. The method of any one of embodiments 1-15, wherein said pentamidine or pharmaceutically acceptable salt thereof is administered to the subject intravenously.

Embodiment 19. The method of any one of embodiments 1-18, wherein said pentamidine or a pharmaceutically acceptable salt thereof is administered to the subject with at least one or more additional anticancer agents.

Embodiment 20. The method of embodiment 19, wherein said additional anticancer agent is etoposide.

Embodiment 21. The method of embodiment 19, wherein said additional anticancer agent is cisplatin.

Embodiment 22. The method of any one of embodiments 1-21, wherein said pentamidine or the pharmaceutically acceptable salt thereof is administered to said subject at a daily dose of about 0.5 mg per kg to about 30 mg per kg.

Embodiment 23. A method of treating a subject suffering from small cell lung cancer, the method comprising
a. assaying a small cell lung cancer sample derived from said subject to determine the level of expression in said sample of at least one biomarker selected from the group of biomarkers listed in Table 1, Table 2 and optionally at least one biomarker from Table 3;
b. detecting a high level of expression of said at least one biomarker in Table 1 and optionally in Table 3 in said sample relative to a normal control;
c. detecting a low level of expression of said at least one biomarker in Table 2 in said sample relative to a normal control; and
d. administering a therapeutically effective amount of pentamidine or a pharmaceutically acceptable salt thereof, to said subject.

Embodiment 24. The method of embodiment 23, wherein said at least one biomarker from Table 1 is INSM1.

Embodiment 25. A kit comprising:
a. a pentamidine or a pharmaceutically acceptable salt thereof;
b. testing materials for predictive gene expression signature of one or more biomarkers for a subtype of small cell lung cancer sensitive to treatment with pentamidine or pharmaceutically acceptable salt thereof; and
c. an instruction for use.

### Further Exemplary Embodiments

Also among the provided embodiments are:

E1. A method of treating a subject suffering from small cell lung cancer expressing a high level of at least one biomarker selected from the group consisting of INSM1, GADD45G, and C1QL1, the method comprising administering a therapeutically effective amount of pentamidine or a pharmaceutically acceptable salt thereof, to the subject.

E2. A method of treating a subject suffering from small cell lung cancer expressing a high level of at least one biomarker selected from the group consisting of INSM1, GADD45G, and C1QL1 and a low level of at least one biomarker selected from the group consisting of STAT6, SMAD3, and RNF43, the method comprising administering a therapeutically effective amount of pentamidine or a pharmaceutically acceptable salt thereof, to the subject.

E3. A method of treating a subject suffering from small cell lung cancer, the method comprising administering a therapeutically effective amount of pentamidine or a pharmaceutically acceptable salt thereof, to the subject, wherein expression of a high level of at least one biomarker selected from the group consisting of INSM1, GADD45G, and C1QL1 by the small cell lung cancer is used as a basis for selecting the subject to receive treatment.

E4. The method of embodiment E3, wherein expression of a low level of at least one biomarker selected from the group consisting of STAT6, SMAD3, and RNF43 by the small cell lung cancer is used as a basis for selecting the subject to receive treatment.

E5. The method of any one of embodiments E1-E4, further comprising determining the level of expression of the at least one biomarker selected from the group consisting of INSM1, GADD45G, and C1QL1.

E6. The method of any one of embodiments E1-E5, further comprising determining the level of expression of at least one biomarker selected from the group consisting of STAT6, SMAD3, and RNF43 by the small cell lung cancer.

E7. A method of treating a subject suffering from small cell lung cancer, the method comprising
a) determining the level of expression by the small cell lung cancer of at least one biomarker selected from the group consisting of INSM1, GADD45G, and C1QL1; and
b) administering a therapeutically effective amount of pentamidine or a pharmaceutically acceptable salt thereof, to said subject, wherein the small cell lung cancer expresses a high level of the at least one biomarker selected from the group consisting of INSM1, GADD45G, and C1QL1.

E8. A method of identifying a subject suitable for small cell lung cancer treatment, the method comprising determining the level of expression of at least one biomarker selected from the group consisting of INSM1, GADD45G, and C1QL1 by the small cell lung cancer, wherein the subject is identified as suitable for small cell lung cancer treatment with a therapeutically effective amount of pentamidine or a pharmaceutically acceptable salt thereof if the small cell lung cancer expresses a high level of the at least one biomarker selected from the group consisting of INSM1, GADD45G, and C1QL1.

E9. The method of any one of embodiments E1-E8, wherein the method comprises determining the level of expression of INSM1.

E10. The method of any one of embodiments E1-E9, wherein the method comprises determining the level of expression of GADD45G.

E11. The method of any one of embodiments E1-E10, wherein the method comprises determining the level of expression of C1QL1.

E12. The method of any one of embodiments E1-E11, wherein the small cell lung cancer expresses a high level of INSM1.

E13. The method of any one of embodiments E1-E12, wherein the small cell lung cancer expresses a high level of GADD45G.

E14. The method of any one of embodiments E1-E13, wherein the small cell lung cancer expresses a high level of C1QL1.

E15. The method of any one of embodiments E1-E15, wherein the high level is a level greater than a control level.

E16. The method of any one of embodiments E7-E15, further comprising determining the level of expression of at least one biomarker selected from the group consisting of STAT6, SMAD3, and RNF43 by the small cell lung cancer.

E17. The method of any one of embodiments E1-E16, wherein the method comprises determining the level of expression of STAT6.

E18. The method of any one of embodiments E1-E17, wherein the method comprises determining the level of expression of SMAD3.

E19. The method of any one of embodiments E1-E18, wherein the method comprises determining the level of expression of RNF43.

E20. The method of any one of embodiments E1, E3, or E5-E19, wherein the small cell lung cancer expresses a low level of at least one biomarker selected from the group consisting of STAT6, SMAD3, and RNF43.

E21. The method of any one of embodiments E1-E20, wherein the small cell lung cancer expresses a low level of STAT6.

E22. The method of any one of embodiments E1-E21, wherein the small cell lung cancer expresses a low level of SMAD3.

E23. The method of any one of embodiments E1-E22, wherein the small cell lung cancer expresses a low level of RNF43.

E24. The method of any one of embodiments E2, E4-E6, and E9-E23, wherein the low level is a level that is lower than a control level or undetectable.

E25. The method of any one of embodiments E1-E24, wherein the small cell lung cancer expresses a high level of expression at least one biomarker from the group consisting of APLP1, SEZ6L2, LHX2, SMAD9, DPF1, NBEA, MPPED1, PCSK1N, WSCD1, RALYL, H2AFX, TUBB3, GABRD, HOXB8, DLX2, CLDN11, CKB, ID4, SBNO2, FZD9, AKR1C3, MTAP, SNCAIP, DLX5, CADPS, DGKB, LFNG, CER1, KIAA0319, VWA5B2, SLC22A17, FOXA2, HNRNPA0, HELLS, NRTN, and SOX21.

E26. The method of any one of embodiments E1-E25, further comprising detecting a high level of expression at least one biomarker from the group consisting of APLP1, SEZ6L2, LHX2, SMAD9, DPF1, NBEA, MPPED1, PCSK1N, WSCD1, RALYL, H2AFX, TUBB3, GABRD, HOXB8, DLX2, CLDN11, CKB, ID4, SBNO2, FZD9, AKR1C3, MTAP, SNCAIP, DLX5, CADPS, DGKB, LFNG, CER1, KIAA0319, VWA5B2, SLC22A17, FOXA2, HNRNPA0, HELLS, NRTN, and SOX21.

E27. The method of any one of embodiments E5-E26, wherein determining the level of expression comprises assaying a nucleic acid or protein level in a small cell lung cancer sample from said subject.

E28. The method of embodiment E27, wherein the small cell lung cancer sample is a fluid sample comprising circulating tumor cells or cell-free nucleic acids.

E29. The method of embodiment E27, wherein the small cell lung cancer sample is a tissue or a cell sample.

E30. The method of any one of embodiments E5-E29, wherein the level of expression of said biomarker is determined by detecting mRNA levels.

E31. The method of any one of claims E5-E29, wherein the level of expression of said biomarkers is determined at the protein level.

E32. The method of claim E31, wherein the presence of the protein is detected using an antibody that binds to the protein.

E33. The method of claim E32, wherein the antibody is labeled.

E34. The method of claim E32 or E33, wherein the level of expression of said biomarker is determined by immunoassay, a western blot assay, immunofluorimetry, ELISA assay, electrochemiluminescence assay, or immunoprecipitation.

E35. The method of any one of claims E1-E34, wherein the pharmaceutically acceptable salt of pentamidine is pentamidine isethionate.

E36. The method of any one of claims E1-E34, wherein the pharmaceutically acceptable salt of pentamidine is pentamidine mesylate.

E37. The method of any one of claims E1-E34, wherein the pharmaceutically acceptable salt of pentamidine is pentamidine gluconate.

E38. The method of any one of claims E1-E37, wherein said small cell lung cancer is Stage I, II or III small cell lung cancer.

E39. The method of any one of claims E1-E38, wherein said subject is a human patient.

E40. The method of any one of claims E1-E39, wherein said pentamidine or pharmaceutically acceptable salt thereof is administered to the subject orally.

E41. The method of any one of claims E1-E40, wherein said pentamidine or pharmaceutically acceptable salt thereof is administered to the subject by inhalation.

E42. The method of any one of claims E1-E40, wherein said pentamidine or pharmaceutically acceptable salt thereof is administered to the subject intravenously.

E43. The method of any one of claims E1-E42, wherein said pentamidine or a pharmaceutically acceptable salt thereof is administered to the subject with at least one or more additional anticancer agents.

E44. The method of claim E43, wherein said additional anticancer agent is etoposide.

E45. The method of claim E43, wherein said additional anticancer agent is cisplatin.

E46. The method of any one of claims E1-E45, wherein said pentamidine or the pharmaceutically acceptable salt thereof is administered to said subject at a daily dose of about 0.5 mg per kg to about 30 mg per kg.

E47. A kit comprising:
a) a pentamidine or a pharmaceutically acceptable salt thereof;
b) testing materials for predictive gene expression signature of one or more biomarkers for a subtype of small cell lung cancer sensitive to treatment with pentamidine or pharmaceutically acceptable salt thereof; and
c) an instruction for use.

The present invention is further illustrated by the following examples which should not be construed as further limiting. The contents of all references, patents and published patent applications cited throughout this application, as well as the Figures and the Appendix of sequences provided herein, are expressly incorporated herein by reference in their entirety.

### Examples

### Example 1. Identification of Pentamidine Susceptibility Biomarkers

A computational model was constructed to identify patient subpopulations of small cell lung cancer, leveraging data from 5 different datasets of treatment-naïve human small cell lung cancer biopsies (Gene Expression Omnibus identifiers GSE30219, GSE43346, GSE50451; George et al. (2015) Nature 524:47-53; and Rudin et al. (2012) Nat. Genet. 44:1111-1116). Pentamidine was a strong match to one SCLC subpopulation identified by the model. Potential biomarker genes for each subpopulation were discovered using another modeling approach. These genes were then filtered by selecting biomarker genes whose expression were significantly altered after dosing of pentamidine in human small cell lung cancer patient derived xenograft (SCLC PDX) study. This approach resulted in identification of more than 40 genes which strongly exhibited a reversed gene expression signature from that of a SCLC tumor in response to treatment with pentamidine. Among the 41 genes (see Tables 1, 2 and 3), the following six genes were selected for further study: INSM1, GADD45G, C1QL1, STAT6, SMAD3 and RNF43.

For all possible combinations of these six genes, we fitted a logistic regression model (Sperandei (2014) Biochem Med (Zagreb) 24:12-18), a Naïve Bayes classifier (Hand (2009) The Top Ten Algorithms in Data Mining, eds. Wu X., Kumar V (London: Chapman Hall), pp 163-178), and a decision tree classifier (Kingsford et al. (2008) Nat. Biotechnol. 26:1011-1013) to gene expression data from the tumors. To ensure comparable and replicable results in the face of batch effects, the data was rescaled and performance was re-evaluated using batch-wise cross-validation as described below. Many of the classifiers had mean precision and recall >0.85 and small confidence intervals for those parameters (see Table 4), suggesting that the biomarkers are strongly associated with membership in the relevant subpopulation of SCLC.

Gene expression data obtained using different measurement platforms were highly prone to batch effects (Leek et al. (2010) Nat Rev Genet 11:733-379), and posed a great complication in cross-dataset comparisons. To ameliorate the batch effects, the gene expression data was re-scaled using the method of Vandesomple and co-workers (Vandesompele et al. (2002) Genome Biol 3:research0034.1). Briefly, a set of housekeeping genes was derived from the literature (Zhu et al. (2008) BMC Genomics 9:172; Cabiati et al. (2012) J Mol Endocrinol 48:251-260; Tilli et al. (2016) BMC Genomics 17:639): ACTB, GAPDH, POLR2A, YWHAG, RPL13A, SDHA, PPIA, TBP, HPRT1, TFRC, PUM1, PGK1, GUSB, DIMT1, TUBA1A, B2M. Three genes in particular, i.e., PPIA, ACTB, RPL13A, showed the most consistent expression (as defined by Vandesompele) across all five datasets and were selected to compute the control expression level. Within each dataset all other genes were scaled by the geometric mean of the three genes. To estimate the classifier performance on future data, leave-one-out cross-validation was performed at the dataset level. Each of the classifiers was fitted five times, each time placing one of the datasets aside and using it to test precision and recall. Shown in Table 4 is the mean precision and recall from all five fittings, as well as their confidence intervals, allowing one to gauge both the expected performance and robustness to noise.

### Example 2. In Vivo Patient Derived Xenograft (PDX) Study

50 mice were enrolled in the study. 25 mice were randomly allocated to each of two human small cell lung cancer tumor models: LU5181 and LU5243. Based on several expression analyses, the expression profile of the human SCLC LU5181 sample was predicted to be a non-responder as it did not correspond to the predicted gene expression signature for pentamidine or a pharmaceutically acceptable salt thereof described herein. However, expression profile of human LU5243, another SCLC tumor sample, successfully matched with the predicted gene expression signature for pentamidine (e.g., as described above, and in Table 1 and Table 2).

Randomization of the 50 mice was performed using the multi-task method in the Study Log software on day 0. Average tumor volume (mm³) for each group ±SD at randomization was as follows. For each LU model, 25 mice were allocated to 4 groups: Group01 Saline (vehicle, n=6), Group02 pentamidine at 20 mg/kg (n=7), Group03 pentamidine at 10 mg/kg (n=7), and Group04 pentamidine at 20 mg/kg (n=5).

Tumor cryovials containing tumor cells were thawed and prepared for inoculation into mice. Cells were washed in PBS, counted, and re-suspended in cold PBS at concentrations of 100,000 viable cells/100 μL. Cell suspensions were mixed with an equal volume of Cultrex ECM and kept on ice during transport to the vivarium. Cells for injections were prepared by withdrawing ECM-Cell mixture into a chilled 1 mL Lure-lok syringe fitted with a 26_{7/8} G (0.5 mm×22 mm) needle. The filled syringes were kept on ice to avoid the solidification of ECM. Animals were prepared for injection using standard approved isoflurane anesthesia. One mouse at a time was immobilized and the site of injection was disinfected with an alcohol swab. 200 μL of the cell suspension in ECM was injected, successfully inoculating approximately 100,000 cells in the right rear flank subcutaneously. Mice were left undisturbed for nine days before observing tumor growth.

Mice were monitored weekly for palpable tumors, or any changes in appearance or behavior. Once tumors were palpable, they were measured twice a week using calipers. Tumor volume was calculated using the following equation (longest diameter×shortest diameter²)/2.

Stock solution of pentamidine was made in water at 10 mg/mL. Amount needed for dosing for one week was calculated and was diluted with PBS. Each animal was dosed with total volume of 10 mL/kg intraperitoneally (IP). Dosing for Group01-03 began on day 1, a day after randomization when average tumors were between 100-200 mm³ (for Groups 1, 2 and 3 only) and continued for 29-33 days. For Group04, five animals that were above randomization range (200-400 mm³) were dosed for 3 days.

TABLE 5
Study Groups and Dosing Regimen for LU5181 (a predicted non-responder).

| | | | | Dosing Frequency | | Dose Volume |
|---|---|---|---|---|---|---|
| Group | Treatment | N | Route | & Duration | (mg/kg) | (mL/kg) |
| 1 | Vehicle | 6 | IP | QDx29 | N/A | 10 |
| 2 | Pentamidine | 7 | IP | QDx29 | 20 | 10 |
| 3 | Pentamidine | 7 | IP | QDx29 | 10 | 10 |
| 4 | Pentamidine | 5 | IP | QDx3 | 20 | 10 |

TABLE 6
Study Groups and Dosing Regimen for LU5243 (a predicted responder).

| | | | | Dosing Frequency | Dose | Dose Volume |
|---|---|---|---|---|---|---|
| Group | Treatment | N | Route | & Duration | (mg/kg) | (mL/kg) |
| 1 | Vehicle | 6 | IP | QDx33 | N/A | 10 |
| 2 | Pentamidine | 7 | IP | QDx30 | 20 | 10 |
| 3 | Pentamidine | 7 | IP | QDx33 | 10 | 10 |
| 4 | Pentamidine | 5 | IP | QDx3 | 20 | 10 |

Body weight was measured twice a week. All measurements were performed prior to dosing of test articles on day of measurement during the treatment period.

For all animals, tumors were collected, weighed, weight recorded and snap frozen. Blood for plasma isolation was taken from each animal at 4 hours after dosing. All samples collected from this study were stored in −80° C. For Group01-03, any time that an animal has to be taken down prior to normal end of study termination, the animal was dosed and sampled 4 hours after the dose was given. For group04, tumors were collected 4 hours after the third dose was given. Tumors were weighed and snap-frozen.

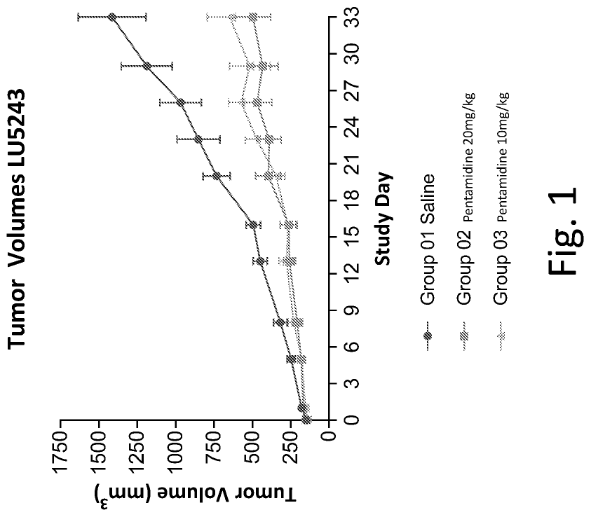

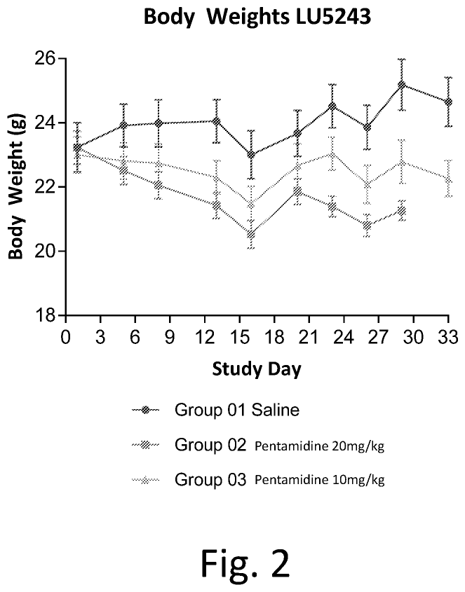

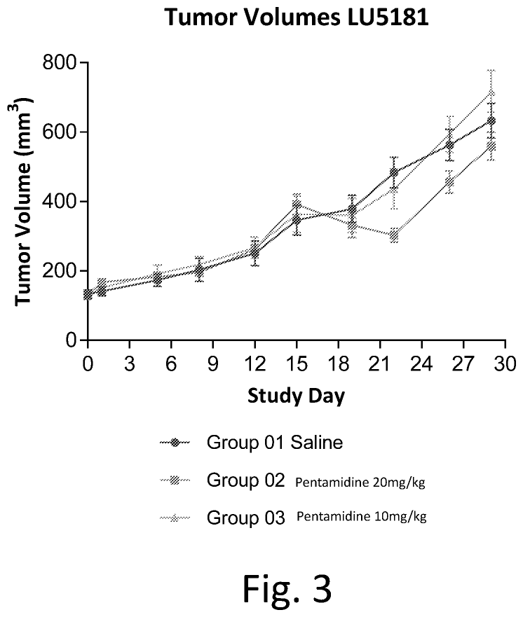

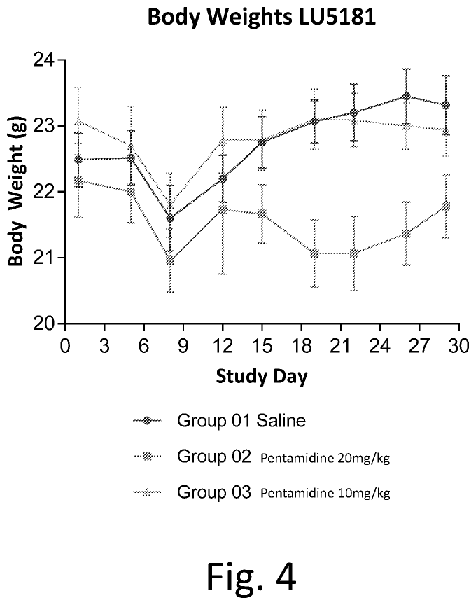

Pentamidine showed significant efficacy in a PDX model for human small cell lung cancer sample LU5243 (a predicted responder) (FIGS. 1 and 2), but not in LU5181 (a predicted non-responder) (FIGS. 3 and 4).

Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled. Such modifications are intended to fall within the scope of the appended claims.

All references, patent and non-patent, cited herein are incorporated herein by reference in their entireties and for all purposes to the same extent as if each individual publication or patent or patent application was specifically and individually indicated to be incorporated by reference in its entirety for all purposes.

What is claimed is:

1. A method of treating a subject suffering from small cell lung cancer expressing a high level of at least one biomarker selected from the group consisting of INSM1, GADD45G, and C1QL1, the method comprising administering a therapeutically effective amount of pentamidine or a pharmaceutically acceptable salt thereof, to the subject.

2. A method of treating a subject suffering from small cell lung cancer expressing a high level of at least one biomarker selected from the group consisting of INSM1, GADD45G, and C1QL1 and a low level of at least one biomarker selected from the group consisting of STAT6, SMAD3, and RNF43, the method comprising administering a therapeutically effective amount of pentamidine or a pharmaceutically acceptable salt thereof, to the subject.

3. A method of treating a subject suffering from small cell lung cancer, the method comprising
administering a therapeutically effective amount of pentamidine or a pharmaceutically acceptable salt thereof, to the subject, wherein expression of a high level of at least one biomarker selected from the group consisting of INSM1, GADD45G, and C1QL1 by the small cell lung cancer is used as a basis for selecting the subject to receive treatment.

4. The method of claim 3, wherein expression of a low level of at least one biomarker selected from the group consisting of STAT6, SMAD3, and RNF43 by the small cell lung cancer is used as a basis for selecting the subject to receive treatment.

5. The method of any one of claims 1-4, further comprising determining the level of expression of the at least one biomarker selected from the group consisting of INSM1, GADD45G, and C1QL1.

6. The method of any one of claims 1-5, further comprising determining the level of expression of at least one biomarker selected from the group consisting of STAT6, SMAD3, and RNF43 by the small cell lung cancer.

7. A method of treating a subject suffering from small cell lung cancer, the method comprising
a) determining the level of expression by the small cell lung cancer of at least one biomarker selected from the group consisting of INSM1, GADD45G, and C1QL1; and
b) administering a therapeutically effective amount of pentamidine or a pharmaceutically acceptable salt thereof, to said subject, wherein the small cell lung cancer expresses a high level of the at least one biomarker selected from the group consisting of INSM1, GADD45G, and C1QL1.

8. A method of identifying a subject suitable for small cell lung cancer treatment, the method comprising determining the level of expression of at least one biomarker selected from the group consisting of INSM1, GADD45G, and C1QL1 by the small cell lung cancer, wherein the subject is identified as suitable for small cell lung cancer treatment with a therapeutically effective amount of pentamidine or a pharmaceutically acceptable salt thereof if the small cell lung cancer expresses a high level of the at least one biomarker selected from the group consisting of INSM1, GADD45G, and C1QL1.

9. The method of any one of claims 1-8, wherein the method comprises determining the level of expression of INSM1.

10. The method of any one of claims 1-9, wherein the method comprises determining the level of expression of GADD45G.

11. The method of any one of claims 1-10, wherein the method comprises determining the level of expression of C1QL1.

12. The method of any one of claims 1-11, wherein the small cell lung cancer expresses a high level of INSM1.

13. The method of any one of claims 1-12, wherein the small cell lung cancer expresses a high level of GADD45G.

14. The method of any one of claims 1-13, wherein the small cell lung cancer expresses a high level of C1QL1.

15. The method of any one of claims 1-14, wherein the high level is a level greater than a control level.

16. The method of any one of claims 7-15, further comprising determining the level of expression of at least one biomarker selected from the group consisting of STAT6, SMAD3, and RNF43 by the small cell lung cancer.

17. The method of any one of claims 1-16, wherein the method comprises determining the level of expression of STAT6.

18. The method of any one of claims 1-17, wherein the method comprises determining the level of expression of SMAD3.

19. The method of any one of claims 1-18, wherein the method comprises determining the level of expression of RNF43.

20. The method of any one of claim 1, 3, or 5-19, wherein the small cell lung cancer expresses a low level of at least one biomarker selected from the group consisting of STAT6, SMAD3, and RNF43.

21. The method of any one of claims 1-20, wherein the small cell lung cancer expresses a low level of STAT6.

22. The method of any one of claims 1-21, wherein the small cell lung cancer expresses a low level of SMAD3.

23. The method of any one of claims 1-22, wherein the small cell lung cancer expresses a low level of RNF43.

24. The method of any one of claims 2, 4-6, and 9-23, wherein the low level is a level that is lower than a control level or undetectable.

25. The method of any one of claims 1-24, wherein the small cell lung cancer expresses a high level of expression at least one biomarker from the group consisting of APLP1, SEZ6L2, LHX2, SMAD9, DPF1, NBEA, MPPED1, PCSK1N, WSCD1, RALYL, H2AFX, TUBB3, GABRD, HOXB8, DLX2, CLDN11, CKB, ID4, SBNO2, FZD9, AKR1C3, MTAP, SNCAIP, DLX5, CADPS, DGKB, LFNG, CER1, KIAA0319, VWA5B2, SLC22A17, FOXA2, HNRNPA0, HELLS, NRTN, and SOX21.

26. The method of any one of claims 1-25, further comprising detecting a high level of expression at least one biomarker from the group consisting of APLP1, SEZ6L2, LHX2, SMAD9, DPF1, NBEA, MPPED1, PCSK1N, WSCD1, RALYL, H2AFX, TUBB3, GABRD, HOXB8, DLX2, CLDN11, CKB, ID4, SBNO2, FZD9, AKR1C3, MTAP, SNCAIP, DLX5, CADPS, DGKB, LFNG, CER1, KIAA0319, VWA5B2, SLC22A17, FOXA2, HNRNPA0, HELLS, NRTN, and SOX21.

27. The method of any one of claims 5-26, wherein determining the level of expression comprises assaying a nucleic acid or protein level in a small cell lung cancer sample from said subject.

28. The method of claim 27, wherein the small cell lung cancer sample is a fluid sample comprising circulating tumor cells or cell-free nucleic acids.

29. The method of claim 27, wherein the small cell lung cancer sample is a tissue or a cell sample.

30. The method of any one of claims 5-29, wherein the level of expression of said biomarker is determined by detecting mRNA levels.

31. The method of any one of claims 5-29, wherein the level of expression of said biomarkers is determined at the protein level.

32. The method of claim 31, wherein the presence of the protein is detected using an antibody that binds to the protein.

33. The method of claim 32, wherein the antibody is labeled.

34. The method of claim 32 or 33, wherein the level of expression of said biomarker is determined by immunoassay, a western blot assay, immunofluorimetry, ELISA assay, electrochemiluminescence assay, or immunoprecipitation.

35. The method of any one of claims 1-34, wherein the pharmaceutically acceptable salt of pentamidine is pentamidine isethionate.

36. The method of any one of claims 1-34, wherein the pharmaceutically acceptable salt of pentamidine is pentamidine mesylate.

37. The method of any one of claims 1-34, wherein the pharmaceutically acceptable salt of pentamidine is pentamidine gluconate.

38. The method of any one of claims 1-37, wherein said small cell lung cancer is Stage I, II or III small cell lung cancer.

39. The method of any one of claims 1-38, wherein said subject is a human patient.

40. The method of any one of claims 1-39, wherein said pentamidine or pharmaceutically acceptable salt thereof is administered to the subject orally.

41. The method of any one of claims 1-40, wherein said pentamidine or pharmaceutically acceptable salt thereof is administered to the subject by inhalation.

42. The method of any one of claims 1-40, wherein said pentamidine or pharmaceutically acceptable salt thereof is administered to the subject intravenously.

43. The method of any one of claims 1-42, wherein said pentamidine or a pharmaceutically acceptable salt thereof is administered to the subject with at least one or more additional anticancer agents.

44. The method of claim 43, wherein said additional anticancer agent is etoposide.

45. The method of claim 43, wherein said additional anticancer agent is cisplatin.

46. The method of any one of claims 1-45, wherein said pentamidine or the pharmaceutically acceptable salt thereof is administered to said subject at a daily dose of about 0.5 mg per kg to about 30 mg per kg.

47. A kit comprising:
a) a pentamidine or a pharmaceutically acceptable salt thereof;
b) testing materials for predictive gene expression signature of one or more biomarkers for a subtype of small cell lung cancer sensitive to treatment with pentamidine or pharmaceutically acceptable salt thereof; and
c) an instruction for use.
